(19) United States
(12) Patent Application Publication
(10) Pub. No.: US 2017/0311599 A1
(43) Pub. Date: Nov. 2, 2017

(54) Target for Anthelmintic Development, and Anthelmintics Utilizing the Same

(71) Applicant: The University of Toledo, Toledo, OH (US)

(72) Inventors: Richard Komuniecki, Toledo, OH (US); Bruce Bamber, Toledo, OH (US); Paul W. Erhardt, Toledo, OH (US)

(73) Assignee: The University of Toledo, Toledo, OH (US)

(21) Appl. No.: 15/581,141

(22) Filed: Apr. 28, 2017

Related U.S. Application Data
(60) Provisional application No. 62/328,819, filed on Apr. 28, 2016.

(51) Int. Cl. A01N 43/40; A01N 43/38; A01K 67/033; A61K 31/4439; A61K 31/404

(52) U.S. Cl. CPC A01N 43/40; A61K 31/4439; A61K 31/404; A01K 67/0336; A01N 43/38; A01K 2217/072; A01K 2227/703

(57) ABSTRACT

Compounds, compositions, methods, materials, and transgenic animals for antihelmintic purposes are described.

### RELATED APPLICATIONS

This application claims priority to U.S. Provisional Application No. 62/328,819, filed under 35 U.S.C. §111(b) on Apr. 28, 2016, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND OF THE INVENTION

Parasitic worms infect humans, animals, and crop plants, causing significant suffering and economic loss. Nematode infections cause significant morbidity and contribute significantly to a loss of disability-adjusted life years. For example, soil-transmitted nematodes, including Necator americanus, Trichuris trichuris, and Ascaris lumbricoides infect nearly 2 billion worldwide and are a source of disease in over 400 million children. In many cases, such as filarial infection, effective chemotherapy is still not available. Parasitic nematodes also have a devastating economic impact in agricultural settings that, at least secondarily, contributes significantly to a decline in human welfare, especially in areas where good nutrition is already compromised. For example, parasitic nematodes infect livestock and major crops (corn and soybeans) and cause billions in economic losses yearly in the US alone. Most commercially available anthelmintics have become increasingly ineffective because of growing resistance (benzimidazoles, levamisole, and, most recently, ivermectin) and most nematicides (e.g., DCBP (1,2-dibromo-3-chloropropane), methyl bromide) to control plant nematodes have been banned by the EPA because of human toxicity. New drugs, new drug targets, and new, more effective screening protocols are desperately needed in all settings. There is a need especially for new drugs to combat emerging resistance to current anthelmintics.

Most anthelmintics in use today act as agonists at key receptors and cause paralysis by interfering with muscle contraction and/or locomotion. Since receptor “activation” is important for anthelmintic activity, receptor knockout is not necessarily the “gold standard” for target validation; in fact, knockout may not be lethal. Five molecular targets have been used for drug discovery: two nicotinic cholinergic receptor subunits (tetrahydropyrimidines/imidathiazoles, and amino-acetonitriles), glutamate-/GABA-gated Cl⁻ channels (macrocyclic lactones and piperazine, respectively) and Ca⁺⁺-gated K⁺ channels (emodepside). The identification of new targets has been limited by the lack of useful information about the identity, function, and localization of the additional receptors regulating muscle contraction and locomotion. In addition to identifying new targets, new screening protocols that preserve the unique pharmacologies of the receptors from the different parasites and maintain a nematode-specific context that includes the cuticle and appropriate accessory proteins are also needed, especially given that no nematode cells lines are available and that the parasites themselves are extremely difficult and expensive to culture.

### SUMMARY OF THE INVENTION

Provided is a method of causing locomotory paralysis in a nematode leading to dysfunctional behavior or death, the method comprising exposing the nematode to an effective amount of one or more compounds taken from the family encompassed by Formula I:

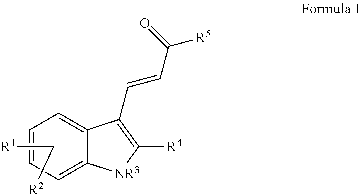

where R¹ and R² are either together or independently H, OH, or O-alkyl; R³ and R⁴ are either together or independently H, alkyl or aralkyl; and R⁵ is alkyl, aryl or aralkyl, where aryl includes heteroaryl and can be further substituted with one or more identical or different alkyl, O-alkyl, or OH groups; and where the family's various salts, stereoisomers, hydrates, solvates, racemates, polymorphs, and simple prodrug forms are also included.

In certain embodiments, R¹ is an OH located at position 5 on the indole ring; R² is H; R³ and R⁴ are either together or independently H, alkyl, or aralkyl; and R⁵ is a phenyl-group or a phenyl-group further substituted with 1 to 3 identical or different OH or OCH₃ groups. In certain embodiments, R¹ is an OH located at position 5 on the indole ring; R² and R³ are H; R⁴ is H, alkyl, or aralkyl; and R⁵ is a pyridine group attached either ortho-, meta-, or para- to the carbonyl moiety.

In certain embodiments, R⁵ is either phenyl or pyridinyl, one of R¹ and R² is H, and the other of R¹ and R² is H, OH, or O-alkyl. In particular embodiments, R³ is H, and R⁴ is either H or alkyl.

In certain embodiments, the compound is compound CD3-718:

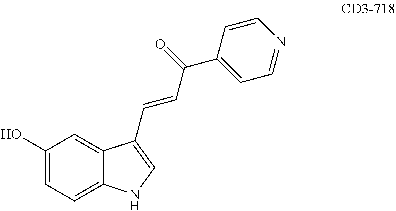 [SMILES: O=C(/C=C/c1c[nH]c2ccc(O)cc12)c1ccncc1]

In certain embodiments, the compound is compound CD3-664:

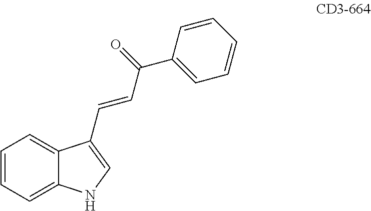 [SMILES: O=C(/C=C/c1c[nH]c2ccccc12)c1ccccc1]

In certain embodiments, the compound is compound CD4:

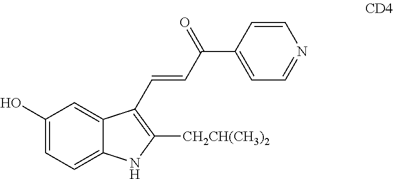

In certain embodiments, the compound is a compound comprising Formula II:

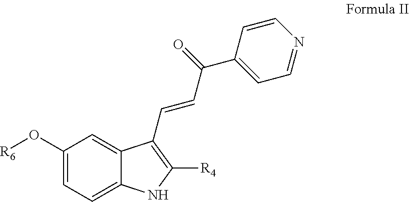

wherein R₄ and R₆ are each independently H or alkyl.

In particular embodiments, the compound is compound CD3-719:

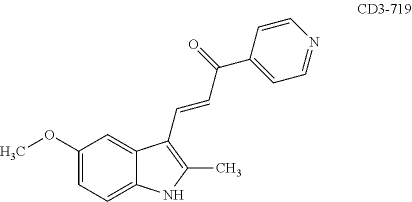 [SMILES: COc1ccc2[nH]c(C)c(/C=C/C(=O)c3ccncc3)c2c1]

In particular embodiments, the compound is CD3-980:

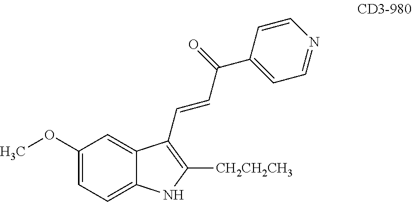

In particular embodiments, the compound is CD3-984:

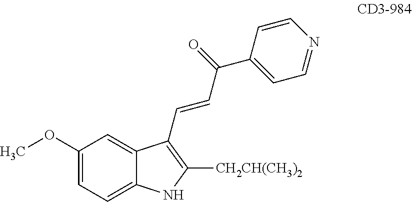

In certain embodiments, the nematode is present in the soil or water of a selected environment such as a farm or a drinking water supply. In certain embodiments, the nematode is present within the physical quarters of other living species such as plant greenhouses, animal barns, or human homes and public buildings. In certain embodiments, the nematode is present within the physical operations of manufacturing plants or commercial buildings such as restaurants or grocery stores.

Also provided is a method of treating a nematode infection in a living host, the method comprising administering to the host an effective amount of one or more compounds in the family of compounds encompassed by Formula I, and treating the nematode infection. In certain embodiments, R⁵ is either phenyl or pyridinyl, one of R¹ and R² is H, and the other of R¹ and R² is H, OH, or O-alkyl. In particular embodiments, R³ is H, and R⁴ is either H or alkyl. In certain embodiments, the compound comprises Formula II. In certain embodiments, the compound is compound CD3-718, compound CD4, compound CD3-664, compound CD3-719, compound CD3-980, or compound CD3-984.

In certain embodiments, R¹ is an OH located at position 5 on the indole ring; R² is H; R³ and R⁴ are either together or independently H, alkyl, or aralkyl; and R⁵ is a phenyl-group or a phenyl-group further substituted with 1 to 3 identical or different OH or OCH₃ groups. In certain embodiments, R¹ is an OH located at position 5 on the indole ring; R² and R³ are H; R⁴ is H, alkyl, or aralkyl; and R⁵ is a pyridine group attached either ortho-, meta-, or para- to the carbonyl moiety. In particular embodiments, R⁴ is H and R⁵ is attached at the para-position.

In certain embodiments, the host is not harmed by the treatment. In certain embodiments, the host is a human, animal, or plant. In certain embodiments, the host is a pig, sheep, horse, cow, goat, dog, cat, chicken, or turkey. In certain embodiments, the host is a soy bean plant or a corn plant. In certain embodiments, the nematode is from a genus selected from the group consisting of: Haemonchus, Trichostrongylus, Ctenocephalides, Dirofilaria, Ostertagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridia, Oxyuris, Ancylostoma, Uncinaria, Toxascaris, Caenorhabditis, Parascaris, Bursaphalenchus, Criconemella, Ditylenchus, Globodera, Helicotylenchus, Heterodera, Meloidogyne, Pratylenchus, Radolpholus, Rotelynchus, Panagrellus, and Tylenchus.

Also provided is a transgenic Caenorhabditis elegans comprising a 5-HT receptor null animal expressing a nematode, insect, animal, or human orthologue of a Gαₒ-coupled 5-HT₁-like receptor in cholinergic motor neurons. In certain embodiments, the Gαₒ-coupled 5-HT₁-like receptor comprises a human 5-HT_{1A} receptor. In certain embodiments, the Gαₒ-coupled 5-HT₁-like receptor comprises nematode SER-4.

Also provided is a method of assaying for anthelmintic selectivity, the method comprising administering a compound to a transgenic Caenorhabditis elegans described herein that expresses a human orthologue of the Gαₒ-coupled 5-HT₁-like receptor, and observing whether the transgenic Caenorhabditis elegans exhibits locomotory paralysis; and administering the compound to a wild type Caenorhabditis elegans or to a Caenorhabditis elegans expressing a nematode orthologue of the Gαₒ-coupled 5-HT₁-like receptor, and observing whether the wild type Caenorhabditis elegans or the elegans expressing a nematode orthologue of the Gαₒ-coupled 5-HT₁-like receptor exhibits locomotory paralysis to determine if the compound has selective antihelmintic activity, where locomotory paralysis in the wild type Caenorhabditis elegans or the Caenorhabditis elegans expressing the nematode orthologue of the Gαₒ-coupled 5-HT₁-like receptor, and a lack or decreased amount of locomotory paralysis in the transgenic Caenorhabditis elegans, is indicative of selective antihelmintic activity.

Also provided is a composition comprising a nematicidally effective amount of a compound encompassed by Formula I, or a salt, stereoisomer, racemate, hydrate, polymorph, or prodrug thereof; and an animal feed premix or supplement. In certain embodiments, the compound comprises Formula II. In certain embodiments, the compound is compound CD3-718, compound CD3-664, compound CD4, compound CD3-719, compound CD3-980, or compound CD3-984. Further provided is a method of treating, preventing, or ameliorating a parasitic infection in animals, the method comprising feeding this composition to animals, and treating, preventing, or ameliorating a parasitic infection in the animals. In certain embodiments, the parasitic infection comprises heartworm (Dirofilaria immitis).

Also provided is a method of treating, preventing, or ameliorating a parasitic infection in animals, the method comprising dispersing an effective amount of a compound encompassed by Formula I, or a salt, stereoisomer, racemate, hydrate, polymorph, or prodrug thereof, into feed or water; and feeding the feed or water to animals to treat, prevent, or ameliorate a parasitic infection in the animals. In certain embodiments, the compound comprises Formula II. In certain embodiments, the compound is compound CD3-718, compound CD3-664, compound CD4, compound CD3-719, compound CD3-980, or compound CD3-984. In certain embodiments, the parasitic infection comprises heartworm (Dirofilaria immitis).

Also provided is a method of screening for anthelmintic compounds, the method comprising expressing a parasitic nematode drug target in a Caenorhabditic elegans worm, and testing a compound for an ability to cause paralysis or locomotory confusion in the worm by activating the drug target, and thereby screening for anthelmintic compounds. In certain embodiments, the parasitic nematode drug target comprises a Gαₒ-coupled 5-HT₁-like receptor. In certain embodiments, the parasitic nematode drug target comprises SER-4. In certain embodiments, the compound is tested for paralytic activity in one or more additional worm strains, wherein the one or more additional worm strains is selected from the group consisting of: native C. elegans, C. elegans lacking all 5-HT receptors, and C. elegans expressing the human 5-HT_{1A} receptor. In particular embodiments, the method further comprises structurally modifying a compound which paralyzes the Caenorhabditic elegans worm expressing the parasitic nematode drug target but does not paralyze the one or more additional worm strains. In certain embodiments, the Caenorhabditic elegans worm is incubated in a hypotonic solution to increase cuticular permeability.

Also provided is a method of treating, preventing, or ameliorating heartworm in a human or animal subject, the method comprising administering to a human or animal subject an effective amount of a compound of Formula I:

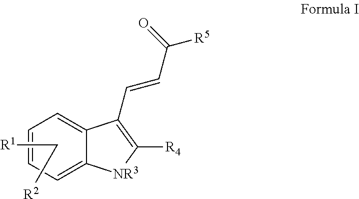

to treat, prevent, or ameliorate heartworm in the subject, wherein R¹, R², R³, and R⁴ are each H, and R⁵ is aryl.

Also provided is a kit for making an anti-parasitic animal feed, the kit comprising a first container housing a composition comprising a compound encompassed by Formula I, or a salt, stereoisomer, racemate, hydrate, polymorph, or prodrug thereof; and a second container housing an animal feed premix or supplement. In certain embodiments, the compound comprises Formula II. In certain embodiments, the compound is compound CD3-718, compound CD3-664, compound CD4, compound CD3-719, compound CD3-980, or compound CD3-984

Also provided are plant seeds comprising a compound encompassed by Formula I, or a salt, stereoisomer, racemate, hydrate, solvate, polymorph, or prodrug thereof. In certain embodiments, the compound comprises Formula II. In certain embodiments, the compound is compound CD3-718, compound CD3-664, compound CD4, compound CD3-719, compound CD3-980, or compound CD3-984.

Also provided is a method of producing plant seeds, the method comprising soaking plant seeds in a composition comprising a compound encompassed by Formula I, or a salt, stereoisomer, racemate, hydrate, solvate, polymorph, or prodrug thereof; and packaging the soaked plant seeds. In certain embodiments, the compound comprises Formula II. In certain embodiments, the compound is compound CD3-718, compound CD3-664, compound CD4, compound CD3-719, compound CD3-980, or compound CD3-984.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1A: Illustration of a basic method to discover chemicals which kill parasitic nematodes.

FIG. 1B: Illustration of a method in accordance with the present disclosure, where chimeric laboratory worms are created to efficiently identify selective anthelmintic compounds.

FIGS. 2A-2B: Schemes showing a non-limiting example of the synthetic route for the preparation of compound CD3-718. FIG. 2A depicts an aldol condensation, and FIG. 2B depicts the demethylation of a methoxy precursor.

FIGS. 3A-3F: C. elegans mutants with increased cuticular permeability are hypersensitive to 5-HT-dependent paralysis. FIGS. 3A-3B: Paralysis of wild type and mutant C. elegans on NGM agar plates. FIG. 3A: Wild type animals examined for 5-HT-dependent paralysis as outlined in Example I. Data are presented as mean±SE (n=3). FIG. 3B: Dose-response curves for 5-HT-dependent paralysis on NGM plates at 10 min exposure for wild type and 5-HT quint animals FIGS. 3C-3D: Paralysis of wild type and mutant C. elegans on non-NGM agar (hypotonic) plates. FIG. 3C: Wild type animals were examined for 5-HT-dependent paralysis as outlined in Example I. Data are presented as mean±SE (n=3). FIG. 3D: Dose-response curves for 5-HT-dependent paralysis in hypotonic conditions at 15 min exposure for wild type and 5-HT quint animals. FIGS. 3E-3F: 5-HT-dependent paralysis of wild type and mutant C. elegans on NGM agar plates. FIG. 3E: 5-HT (0.25 mM)-dependent paralysis of wild-type, bus-8 (e2968), bus-16 (e2802), and bus-17 (e2800) mutants. Data are presented as mean±SE (n=3). FIG. 3F: Dose-response curves for 5-HT-dependent paralysis at 10 min exposure for wild type and bus mutants.

FIGS. 4A-4B: The 5-HT/SER-4-dependent inhibition of either the AIB interneurons or cholinergic motor neurons causes locomotory paralysis. FIG. 4A: Confocal images of 5-HT quint expressing SER-4::GFP in the AIB interneurons (Pnpr-9)(A1) or cholinergic motor neurons (Punc-17β)(A2). GFP fluorescence (A2) or GFP fluorescence is overlaid on DIC image (A1). The red stain in A2 is coelomocyte-specific RFP screening marker. FIG. 4B: Paralysis of wild type, mutant and transgenic C. elegans on hypotonic, non-NGM agar plates. Wild type, quadruple 5-HT receptor null animals expressing only SER-4 (SER-4 quad) or 5-HT quint expressing the C. elegans 5-HT₁-like receptor, SER-4, in either the cholinergic motor neurons (Punc-17β) or the two AIB interneurons (Pnpr-9) were examined for 5-HT (1 mM)-dependent paralysis as outlined in Example I. Data are presented as mean±SE (n=3).

FIGS. 5A-5C: 5-HT and 5-HT receptor agonists selectively paralyze C. elegans 5-HT receptor mutant animals expressing nematode, insect, or human 5-HT₁-like receptors in the cholinergic motor neurons. FIGS. 5A-5C: Paralysis of wild type, mutant and transgenic C. elegans on hypotonic, non-NGM agar plates. FIG. 5A: 5-HT (1 mM)-dependent paralysis of 5-HT quint animals expressing either C. elegans 5-HT₁-like (SER-4), Drosophila 5-HT1-like, or human 5-HT1A receptor in cholinergic motor neurons (Punc-17β). Data are presented as mean±SE (n=3). B. 8-OH-DPAT (2 mM)-dependent paralysis of 5-HT quint animals expressing either C. elegans 5-HT₁-like (SER-4), Drosophila 5-HT₁-like, or human 5-HT_{1A} receptor in cholinergic motor neurons (Punc-17β). Data are presented as mean±SE (n=3). FIG. 5C: Sumatriptan (1 mM)-dependent paralysis of wild type, 5-HT quint animals expressing either C. elegans 5-HT₁-like (SER-4), Drosophila 5-HT₁-like, or human 5-HT_{1A} receptor in cholinergic motor neurons (Punc-17β). Data are presented as mean±SE (n=3).

FIGS. 6A-6C: PAPP paralyzes C. elegans via SER-4 and DOP-3. FIGS. 6A-6C: Paralysis of wild type, mutant, and transgenic C. elegans on hypotonic non-NGM agar plates. FIG. 6A: PAPP (0.5 mM)-dependent paralysis of wild-type, 5-HT quint, and 5-HT quint animals expressing SER-4 in the cholinergic motor neurons (Punc-17β). Data are presented as mean±SE (n=3). FIG. 6B: Dose-response curves for PAPP-dependent paralysis at 15 min exposure for wild type, 5-HT quint, and 5-HT quint animals expressing SER-4 in the cholinergic motor neurons (Punc-17β). FIG. 6C: PAPP (0.5 mM)-dependent paralysis of 5-HT quint and 5-HT quint animals expressing Pdop-3::dop-3 RNAi. Data are presented as mean±SE (n=3). ‘*’ p_0.001, significantly different from 5-HT quint animals assayed under identical conditions.

FIGS. 7A-7E: Exogenous monoamines paralyze C. elegans expressing monoamine-gated Cl⁻ channels in either cholinergic motor neurons or body wall muscles. FIG. 7A: Confocal image of 5-HT quint animals expressing H. contortus (Hco) MOD-1::GFP in body wall muscles (Pmyo-3). GFP-fluorescence image. FIGS. 7B-7E: Paralysis of wild type, mutant, and transgenic C. elegans on non-NGM agar plates. FIG. 7B: 5-HT (0.5 mM)-dependent paralysis of wild type, 5-HT quint, and 5-HT quint animals expressing either the C. elegans or H. contortus (Hco) MOD-1 orthologues in the cholinergic motor neurons (Punc-17β) or the H. contortus (Hco) MOD-1 orthologue in body wall muscle (Pmyo-3). Data are presented as mean±SE (n=4). FIG. 7C: Dose-response curves for 5-HT-dependent paralysis at 15 min exposure for wild type, 5-HT quint, and 5-HT quint animals expressing either the C. elegans or H. contortus (Hco) MOD-1 orthologues in the cholinergic motor neurons (Punc-17β) or the H. contortus (Hco) MOD-1 orthologue in body wall muscle (Pmyo-3). FIG. 7D: Tyramine (1 mM)-dependent paralysis of wild type, TA quad, and TA quad animals expressing either the C. elegans LGG-55 in body wall muscle (Pmyo-3) or the H. contortus (Hco) LGC-55 orthologue in the cholinergic motor neurons (Punc-17β). Data are presented as mean±SE (n=3). FIG. 7E: Dose-response curves for TA-dependent paralysis at 15 min exposure for wild type, TA quad, and TA quad animals expressing either LGC-55 in the body wall muscles (Pmyo-3), or H. contortus (Hco) LGC-55 orthologue in cholinergic motor neurons (Punc-17β).

FIGS. 8A-8C: Inhibiting signaling from the two AIB interneurons causes “locomotory confusion” and paralysis. FIGS. 8A-8C: Paralysis of wild type, mutant, and transgenic C. elegans on either NGM or non-NGM agar plates. FIG. 8A: 5-HT quint and 5-HT quint animals expressing MOD-1 in the AIBs (Pinx-1) were examined for 5-HT (1 mM)-dependent paralysis on non-NGM agar plates, as outlined in Example I. Data are presented as mean±SE (n=3). FIGS. 8B-8C: Wild type animals expressing HisCl1 in the AIBs (cx15457) were examined for histamine (2 or 10 mM)-dependent paralysis on NGM (B) and non-NGM (C) agar plates. Data are presented as mean±SE (n=3).

FIGS. 9A-9B: Graphs showing the percent of worms paralyzed after 15 minutes exposed to compounds in C. elegans 5-HT₁ (SER-4) (FIG. 9A) and wild-type (FIG. 9B) worms. Compound CD3-718 exhibited remarkable selectivity.

### DETAILED DESCRIPTION OF THE INVENTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

For convenience, various terms are defined prior to further description of the various embodiments of the present disclosure.

The terms “anthelmintic” or “anthelminthic” refer to an antiparasitic drug that expels parasitic worms (helminths) or other internal parasites by either stunning or killing them without causing significant damage to the host. Anthelmintics are useful for treating humans, animals, and plants that are infected by parasites.

It will be appreciated that any of the compounds described herein may be substituted with any number of substituents or functional moieties. In general, the term “substituted” whether preceded by the term “optionally” or not, and substituents contained in formulas, refer to the replacement of hydrogen atoms in a given structure with a specified substituent. When more than one position in any given structure may be substituted with more than one substituent selected from a specified group, the substituent may be either the same or different at every position.

The term “alkyl” refers to monovalent alkyl groups having from 1 to 50 carbon atoms, preferably having from 1 to 10 carbon atoms, and more preferably having from 1 to 6 carbon atoms. This term is exemplified by groups such as methyl, ethyl, n-propyl, iso-propyl, n-butyl, iso-butyl, n-hexyl, and the like.

The term “aryl” refers to an unsaturated aromatic carbocyclic or heterocyclic group, preferably of from 6 to 14 carbon atoms, having a single ring (e.g., phenyl) or multiple condensed rings (e.g., naphthyl or anthryl), preferably having from 1 to 3 rings. Preferred aryls include phenyl, pyridyl, naphthyl, thienyl, indolyl, and the like. The term “aryl” includes heteroaryl groups. Unless otherwise constrained by the definition for the aryl substituent, such aryl groups can optionally be substituted with from 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, aminocarboxy esters, aralkyl, aryl, aryloxy, carboxyl, carboxylalkyl, acylamino, cyano, halo, nitro, heteroaryl, heterocyclic, oxyacyl, oxyacylamino, thioalkoxy, substituted thioalkoxy, trihalomethyl, mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl, heterocyclic, and the like. Preferred substituents include alkyl, alkoxy, halo, cyano, nitro, trihalomethyl, and thioalkoxy.

The term “aralkyl” refers to an aryl group with an alkyl substitution. Generally, aryalkyl groups herein contain from 6 to 30 carbon atoms. The term “aralkyl” refers to alkylene-aryl groups preferably having from 1 to 10 carbon atoms in the alkylene moiety and from 6 to 10 carbon atoms in the aryl moiety. Such aralyl groups are exemplified by benzyl, phenethyl, and the like.

It will also be appreciated by one of ordinary skill in the art that asymmetric centers may exist in any of the compounds disclosed herein. Thus, the compounds and pharmaceutical compositions thereof may be in the form of an individual enantiomer, diastereomer, or geometric isomer, or may be in the form of a mixture of stereoisomers. In certain embodiments, the compounds are enantiopure compounds. In certain other embodiments, mixtures of stereoisomers or diastereomers are provided. Additionally, the compounds encompass both (Z) and (E) double bond isomers (or cis and trans isomers) unless otherwise specifically designated. Thus, compounds generally depicted in structures herein encompass those structures in which double bonds are (Z) or (E).

The term “solvate” refers to a pharmaceutically acceptable solid form of a specified compound containing solvent molecules as part of the crystal structure. A solvate typically retains at least some of the biological effectiveness of such compound. Solvates can have different solubilities, hygroscopicities, stabilities, and other properties. Examples of solvates include, but are not limited to, compounds in combination with water, isopropanol, ethanol, methanol, DMSO, ethyl acetate, acetic acid, or ethanolamine Solvates are sometimes termed “pseudopolymorphs.”

The term “hydrate” refers to a solvate with water.

The term “racemate” refers to a mixture that contains an equal amount of enantiomers.

The term “polymorph” means a crystalline form of a substance that is distinct from another crystalline form of the substance but that shares the same chemical formula.

The term “prodrug” refers to a precursor or derivative of a particular compound which, when consumed, generates the pharmacologically active compound by action of natural processes or biological conditions. For example, a prodrug can be cleaved, hydrolyzed, or oxidized by enzymes in vivo to produce the pharmacologically active compound.

The term “pharmaceutically acceptable salts,” as used herein, refers to salts of compounds of the present disclosure that are substantially non-toxic to living organisms. Typical pharmaceutically acceptable salts include those salts prepared by reaction of a compound of the present disclosure with an inorganic or organic acid, or an organic base, depending on the substituents present on the compounds.

Examples of inorganic acids which may be used to prepare pharmaceutically acceptable salts include: hydrochloric acid, phosphoric acid, sulfuric acid, hydrobromic acid, hydroiodic acid, phosphorous acid, and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids, and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like. Other suitable salts are known to one of ordinary skill in the art.

Suitable pharmaceutically acceptable salts may also be formed by reacting the compounds described herein with an organic base such as methylamine, ethylamine, ethanolamine, lysine, ornithine, and the like. Other suitable salts are known to one of ordinary skill in the art.

It should be recognized that the particular anion or cation forming a part of any salt is not critical, so long as the salt, as a whole, is pharmacologically acceptable and as long as the anion or cation does not contribute undesired qualities or effects. Further, additional pharmaceutically acceptable salts are known to those skilled in the art, and may be used within the scope of the invention. Additional examples of pharmaceutically acceptable salts and their methods of preparation and use are presented in Pharmaceutical Salts: Properties, Selection and Use—A Handbook, by C. G. Wermuth and P. H. Stahl, Verlag Helvetica Chimica Acta, 2002, which is incorporated herein by reference.

General Description

Provided are anthelmintics, as well as materials and methods for discovering new anthelmintics. In one aspect, the present disclosure provides a method for discovering new compounds with highly selective anti-nematode activity that can be used in medical, veterinary, and agricultural settings with minimal toxicity to the host and environment. In another aspect, chimeric laboratory worms are provided to efficiently identify selective anti-nematode compounds. In another aspect, the present disclosure provides anthelmintic compounds.

Provided are anthelmintic compounds encompassed by the general Formula I:

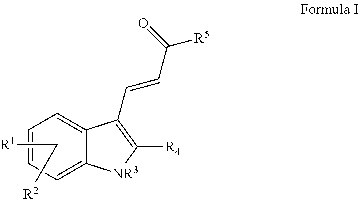

where R¹ and R² are either together or independently H, OH, or O-alkyl; R³ and R⁴ are either together or independently H, alkyl, or aralkyl; and R⁵ is alkyl, aryl, or aralkyl, where aryl includes heteroaryl and can be further substituted with one or more identical or different alkyl, O-alkyl, or OH groups.

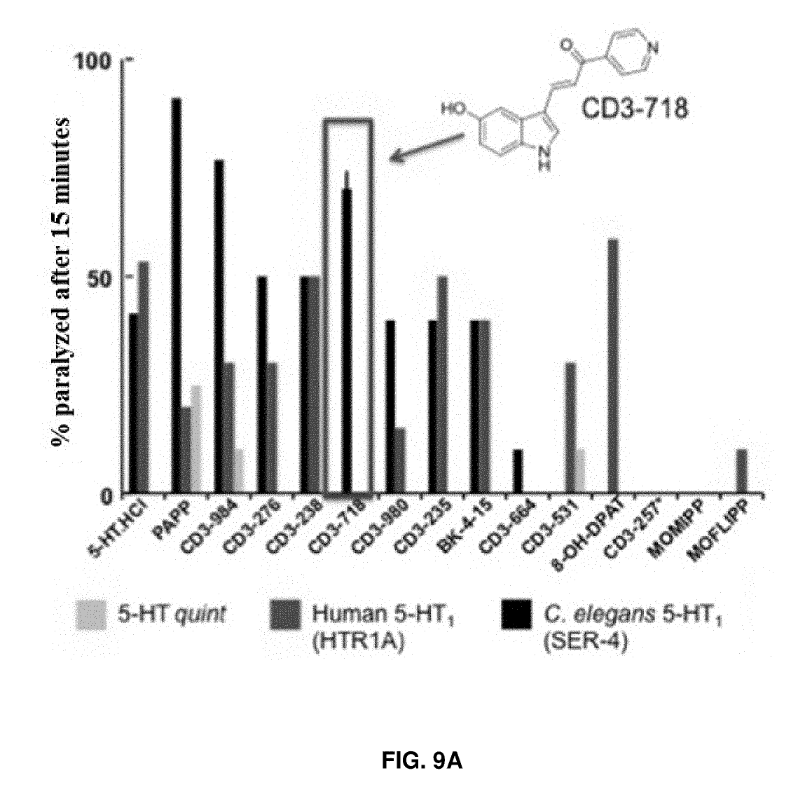

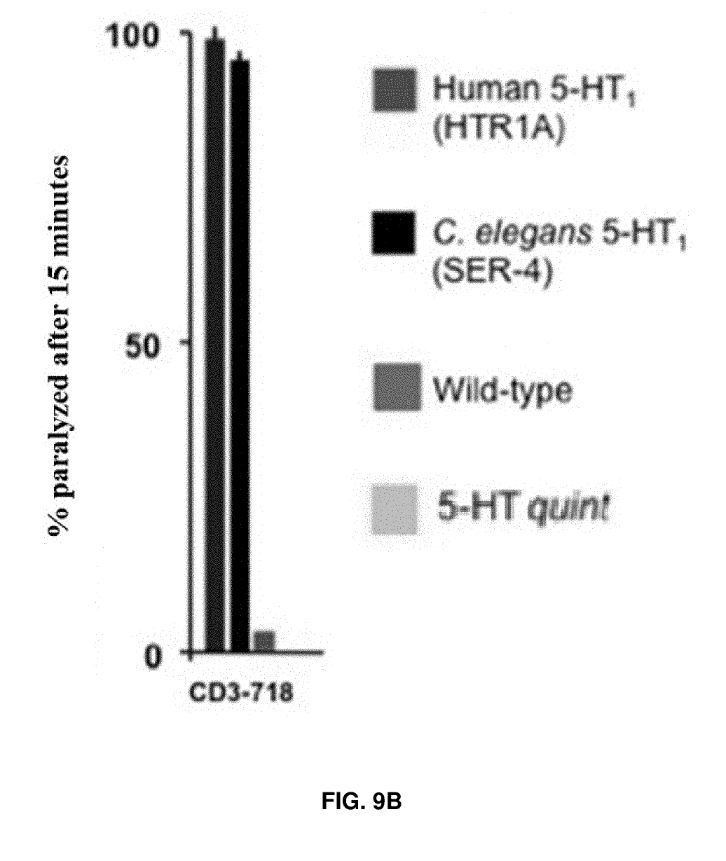

One non-limiting example of a highly selective anti-nematode compound encompassed within Formula I, the selective anti-nematode activity of which was discovered from the method described herein, is a compound referred to as compound CD3-718. (FIGS. 9A-9B.) Compound CD3-718 has the following structural formula:

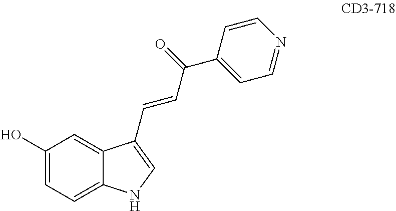 [SMILES: O=C(/C=C/c1c[nH]c2ccc(O)cc12)c1ccncc1]

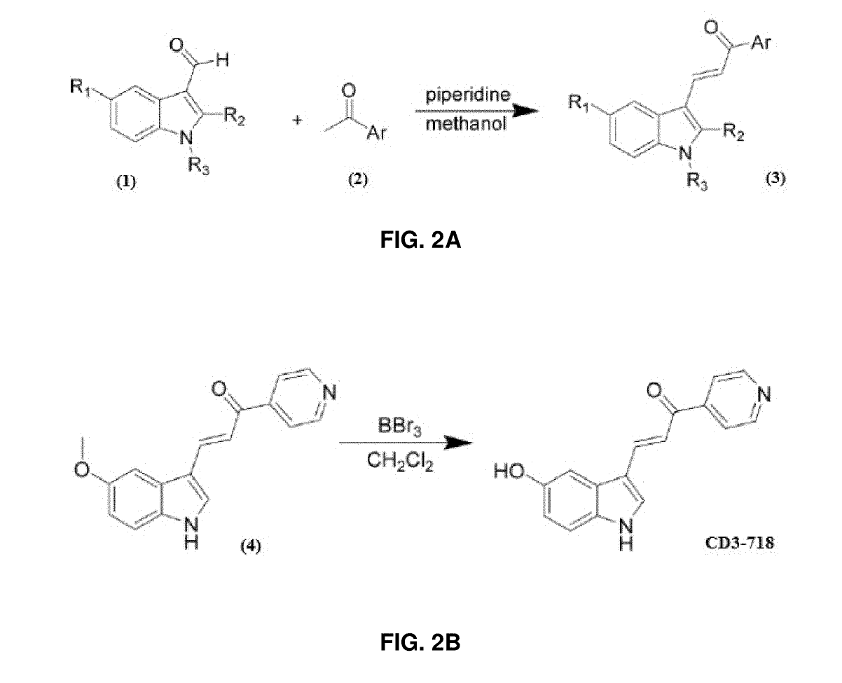

Compound CD3-718 is also known as trans-3-(5-hydroxy-1H-indol-3-yl)-1-(4-pyridinyl)-2-propen-1-one. Compound CD3-718 can be prepared, for example, as described in U.S. Pat. No. 9,028,796, which is incorporated herein by reference in its entirety, and as depicted in FIGS. 2A-2B. Briefly, an aldol condensation between indole-3-carboxaldehyde (1) and an aromatic ketone (2) can be utilized to prepare an indole-3-ketone (3), such as trans-3-(5-methoxy-1H-indol-3-yl)-1-(4-pyridinyl)-2-propen-1-one (4) (where R₁ of compound (3) is methoxy, and R₂ and R₃ of compound (3) are each hydrogen). (FIG. 2A.) Such aldol condensations are known to proceed well employing secondary amines (such as, but not limited to, piperidine) as catalysts. The methoxy ketone (4) can be demethylated, such as with BBr₃, to produce compound CD3-718. (FIG. 2B.) As demonstrated in the Examples herein, compound CD3-718 shows agonist activity that is remarkably selective for the C. elegans 5-HT₁ receptor when compared to the human form. Without wishing to be bound by theory, it is believed that the hydroxyl on the indole ring of CD3-718 may be important for activity.

Another non-limiting example of a compound of Formula I is CD3-664, which has the following structural formula:

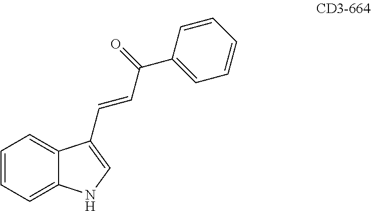 [SMILES: O=C(/C=C/c1c[nH]c2ccccc12)c1ccccc1]

Compound CD3-664 can be prepared, for example, as described in U.S. Pat. No. 9,028,796. Compound CD3-664 has been found to be active against heartworm (Dirofilaria immitis).

Additional non-limiting examples of compounds of Formula I are CD3-719, CD3-980, and CD3-984, which share the general formula of Formula II:

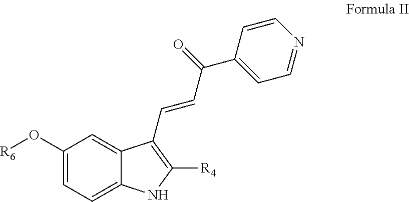

where R₄ and R₆ are each independently H or alkyl. When R₆ is CH₃ and R₄ is CH₃, the compound is CD3-719:

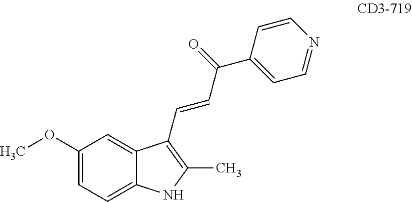 [SMILES: COc1ccc2[nH]c(C)c(/C=C/C(=O)c3ccncc3)c2c1]

When R₆ is CH₃ and R₄ is CH₂CH₂CH₃, the compound is CD3-980:

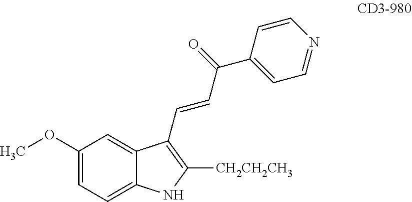

When R₆ is CH₃ and R₄ is CH₂CH(CH₃)₂, the compound is CD3-984:

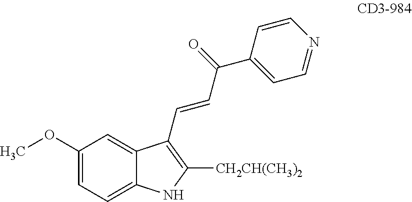

Compounds CD3-719, CD3-980, and CD3-984 can be prepared, for example, as described in U.S. Pat. No. 9,028,796.

Another non-limiting example of a compound of Formula I is compound CD4, which has the following structure:

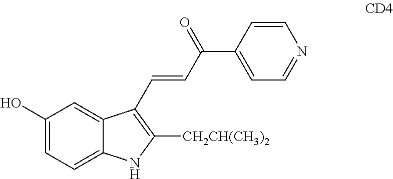

Compound CD4 can also be prepared using the methods described in U.S. Pat. No. 9,028,796.

Compound CD3-719 is similar to compound CD3-718 except that its oxygen analogous to compound CD3-718's hydroxyl on the indole ring is present as a methoxy-substituent. Compounds CD3-980 and CD3-984 have demonstrated at least moderate activity with favorable species selectivity despite having a methoxy-substituent rather than a hydroxyl group at the same position. Without wishing to be bound by theory, it is believed that by increasing hydrophobicity at the R⁴ position of Formula I, the compound's ability to permeate worm cuticle is enhanced. Thus, compound CD3-984 is believed to be able to permeate worm cuticle better than compound CD3-719.

Without wishing to be bound by theory, it is believed that compound CD3-719 has decreased activity due to the substitution of a methoxy for a hydroxyl on the indole ring of CD3-718. Substitution of alkyl groups at the 2-position of the indole ring of CD3-719 (i.e., R⁴ of Formula I) restore activity. Thus, efficacy reflects combined influences of pharmacophore structure and cuticular permeability. Methoxy substitution may reduce biological activity against certain species below detactable levels, due to compromised pharmacophore structure and relatively low cuticular permeability. Alkyl substitutions increase hydrophobicity, and therefore increase cuticular permeability, such that biological activity is restored through greater bioavailability. These conclusions are drawn from observations of nematode locomotion as described in the Examples herein. (FIGS. 9A-9B.)

Compounds of Formula I in general, and compounds CD3-718, CD3-719, CD3-664, CD4, CD3-980, and CD-984 specifically, are useful as anthelmintics. For example, compounds of Formula I, such as compound CD3-718, can be utilized in humans, animals, and plants to treat, prevent, or ameliorate parasitic worm infections. It is understood that various salts, isomers, racemates, polymorphs, and prodrugs of the Formula I compounds are also useable for the same purposes as the Formula I compounds.

For veterinary use, the antihelmintic compound (i.e., a compound of Formula I, such as compound CD3-718, or a salt, isomer, racemate, polymorph, or prodrug thereof) can be added to an animal's diet, or otherwise administered to an animal, in order to treat, prevent, or ameliorate helminthiasis. The group of diseases described generally as helminthiasis is due to infection of an animal host with parastic worms known as helminths. Helminthiasis is a prevalent and serious economic problem in domesticated animals such as swine, sheep, horses, cattle, goats, dogs, cats, and poultry. Among the helminths, the group of worms described as nematodes causes wide-spread and often times serious infection in various species of animals. The most common genera of nematodes infecting the animals referred to above are Haemonchus, Trichostrongylus, Ctenocephalides, Dirofilaria, Ostertagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridia, Oxyuris, Ancylostoma, Uncinaria, Toxascaris, Caenorhabditis, and Parascaris. Certain of these, such as Nematodirus, Cooperia, and Oesophagostomum, attack primarily the intestinal tract, while others, such as Dictyocaulus, are found in the lungs. Still other parasites may be located in other tissues and organs of the body. Considering a pig as a non-limiting example, worms commonly found in pigs are roundworms (Ascaris suum), nodular worms (Oesophagostomum species), intestinal threadworms (Strongyloides randomi), whipworms (Trichuris suis), kidney worms (Stephanurus dentatus), and lungworms (Metastrongylus species).

For use in animals, the anthelmintic compound can be administered orally in a unit dosage form such as a capsule, bolus, or tablet, or as a liquid drench when used as an anthelmintic in mammals. The drench is normally a solution, suspension, or dispersion of the active ingredient, usually in water, together with a suspending agent such as bentonite and a wetting agent or like excipient. The drenches may also contain an antifoaming agent. Drench formulations generally contain from about 0.001 to 0.5% by weight of the active compound. Preferred drench formulations may contain from 0.01 to 0.1% by weight. The capsules and boluses comprise the active ingredient admixed with a carrier vehicle such as starch, talc, magnesium stearate, or dicalcium phosphate.

Where it is desired to administer the anthelmintic compound in a dry, solid unit dosage form, capsules, boluses, or tablets containing the desired amount of active compound can be employed. These dosage forms are prepared by intimately and uniformly mixing the active ingredient with suitable finely divided diluents, fillers, disintegrating agents, and/or binders such as starch, lactose, talc, magnesium stearate, vegetable gums, and the like. Such unit dosage formulations may be varied widely with respect to their total weight and content of the antiparasitic agent, depending upon the factors such as the type of host animal to be treated, the severity and type of infection, and the weight of the host.

When the active compound is to be administered via an animal feedstuff, it is intimately dispersed in the feed or used as a top dressing, or in the form of pellets which may then be added to the finished feed or, optionally, fed separately. Alternatively, the antiparasitic compounds may be administered to animals parenterally, for example, by intraluminal, intramuscular, intratracheal, or subcutaneous injection, in which event the active ingredient is dissolved or dispersed in a liquid carrier vehicle. For parenteral administration, the active material is suitably admixed with an acceptable vehicle, preferably of the vegetable oil variety, such as peanut oil, cotton seed oil, and the like. Other parenteral vehicles, such as organic preparations using solketal, glycerol, formal, and aqueous parenteral formulations, are also used. The active compound or compounds are dissolved or suspended in the parenteral formulation for administration; such formulations generally contain from 0.005 to 5% by weight of the active compound.

When the antihelmintic is administered as a component of the feed of the animals, or dissolved or suspended in the drinking water, compositions are provided in which the active compound or compounds are intimately dispersed in an inert carrier or diluent. By inert carrier is meant one that will not react with the antiparasitic agent and one that may be administered safely to animals. Preferably, a carrier for feed administration is one that is, or may be, an ingredient of the animal ration.

Suitable compositions include feed premixes or supplements in which the active ingredient is present in relatively large amounts and which are suitable for direct feeding to the animal or for addition to the feed either directly or after an intermediate dilution or blending step. Typical carriers or diluents suitable for such compositions include, for example, distillers' dried grains, corn meal, citrus meal, fermentation residues, ground oyster shells, wheat shorts, molasses solubles, corn cob meal, edible bean mill feed, soya grits, crushed limestone, and the like.

For use in humans, the anthelmintic compound can be formulated in a pharmaceutical composition. Pharmaceutical compositions of the present disclosure comprise an effective amount of an anthelmintic described herein (an “active” compound), and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158, 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols, and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this context, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques, which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and laurocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroethylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the anthelmintics described herein are useful for treating, preventing, or ameliorating a parasitic infection. In this regard, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

By way of a non-limiting example of a combination therapy, an anthelmintic compound or composition herein can be administered in combination with one or more suitable other antihelmintic drugs including, but not limited to: albendazole, ivermectin, praziquantel, dichlorvos, fenbadazole, levamisole, piperazine, and pyrantel tartrate.

The antihelmintic compounds herein are also useful as nematicides for the control of soil nematodes and plant parasites, such as those selected from the genera Bursaphalenchus, Criconemella, Ditylenchus, Globodera, Helicotylenchus, Heterodera, Meloidogyne, Pratylenchus, Radolpholus, Rotelynchus, Panagrellus, or Tylenchus. Thus, the anthelmintics are useful in soil amendment compositions. The antihelmintic can be formulated in a nematicidal formulation for use against crop parasites. The antihelmintic can be formulated using standard procedures associated with the use of nematicidal products.

In some embodiments of a nematicidal product, the product is a solution or spray. As would be readily appreciated by a person skilled in the art, the delivery of such a product can be calculated in terms of the active ingredient applied per unit area. For example, the nematicidal product may be applied at a rate of about 0.02 lb/acre to about 0.1 lb/acre, or from about 0.5 lb/acre to about 2 lbs/acre. Similarly, the nematicidal product can be applied at a rate of up to about 16 oz. of formulated product per acre, or from about 4 oz. to about 8 oz. formulated product per acre. A person of ordinary skill in the art would readily appreciate that the desired application rate of the active ingredient could be achieved using a great variety of different concentrations of active ingredient while varying the application rate of the solution. Thus, a large quantity of dilute solution could be applied or a smaller quantity of a more concentrated solution could be applied.

In some embodiments, a nematicidal product described herein can be used to treat a water supply, a building, or some other environment, such as an animal barn, a plant greenhouse, a human home, a public building, a manufacturing plant, a restaurant, or a grocery store.

The antihelmintic can also be dissolved and administered to plants in the form a solution in which plant seeds are soaked prior to packaging or planting. Seed soaking is an effective method for delivering an anthelmintic to plants. A wide variety of suitable solvents, including water, are possible for dissolving the anthelmintic to form a solution in which plant seeds can be soaked.

Alternatively, an anthelmintic formulation can be applied to the soil, where it can be absorbed by crop roots, and then transported to the stems and/or leaves of the crops, so that the whole plant benefits from nematicidal activity.

The tonicity of any medium containing the anthelmintic compound can be decreased to facilitate an easier entrance of the anthelmintic compound into the parasites. Animal parasite cuticle, such as the cutical of gut-dwelling nematodes, is normally more permeable than free-living animal cutical.

It is envisioned that the compounds, compositions, and methods described herein could be embodied as parts of a kit or kits. A non-limiting example of such a kit is a kit for preparing an anti-parasitic animal feed, which includes an effective amount of a compound of Formula I and an animal feed premix or supplement in separate containers, where the containers may or may not be present in a combined configuration. Many other kits are possible, such as kits further comprising a pharmaceutically acceptable carrier, diluent, or excipient, or further comprising a suitable other anthelmintic drug for a combination therapy. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

Further provided is a method of determining coverage of health insurance reimbursement or payments, the method comprising denying coverage or reimbursement for a treatment, wherein the treatment comprises an antihelmintic compound of Formula I.

The challenge with any method of discovering nematicides is how to achieve selectivity and efficiency while maintaining the nematode context. Most proteins are shared between worms and humans, but their chemical sensitivities are unique. Toxicity to humans, animals, and the environment, should be minimized. As one example, compound CD3-718 is remarkably selective for its nematode target. (FIG. 9A.)

Locomotory paralysis is the preferred endpoint of most nematicides. Serotonin (5-HT) rapidly paralyzes both free-living and parasitic nematodes, including the economically significant plant parasites Heterodera glycines (soybean cyst nematode) and Meloidogyne incognita (Southern root-knot nematode). The key 5-HT receptor responsible for this paralysis has been identified and localized. 5-HT itself would not make a good nematicide for a variety of reasons, but it is useful for developing compounds with favorable nematicidal properties (e.g., useful selectivity range, limited environmental toxicity, optimal stability, etc.). Libraries of thousands of ‘serotonin-like’ compounds have been generated to identify specific ligands for individual human 5-HT receptor subtypes.

Since plant parasitic nematodes are difficult to grow in large quantities for screening, a method using the free-living nematode Caenorhabditic elegans has been developed through genetic engineering to express specific receptors from the parasites in mutant backgrounds at sites amenable to high throughput locomotory screening protocols. Nematodes are a diverse group, and expressing the receptor from the appropriate parasite is important to account for any potential pharmacological differences among orthologous receptors. As one non-limiting example, human 5-HT_{1A} receptors have been expressed in C. elegans, which faithfully displays human pharmacology in the nematode context.

In another example, M. incognita 5-HT receptors can be expressed in a genetically-engineered C. elegans mutant background. Possible 5-HT-like ligands can be screened against these receptors. M. incognita contains a clear ortholog of SER-4, the 5-HT_{1A}-like receptor from C. elegans that causes 5-HT-dependent paralysis. cDNA pools can be synthesized from M. incognita J2 larval RNA, and Mi-ser-4 cDNA can be isolated by PCR and RACE cloning, and validated in heterologous expression assays. Expression constructs can be injected into C. elegans lacking endogenous 5-HT receptors. Transgenic worms can be tested for Mi-SER-4 expression using 5-HT paralysis in locomotion assays, and direct electrophysiological assays.

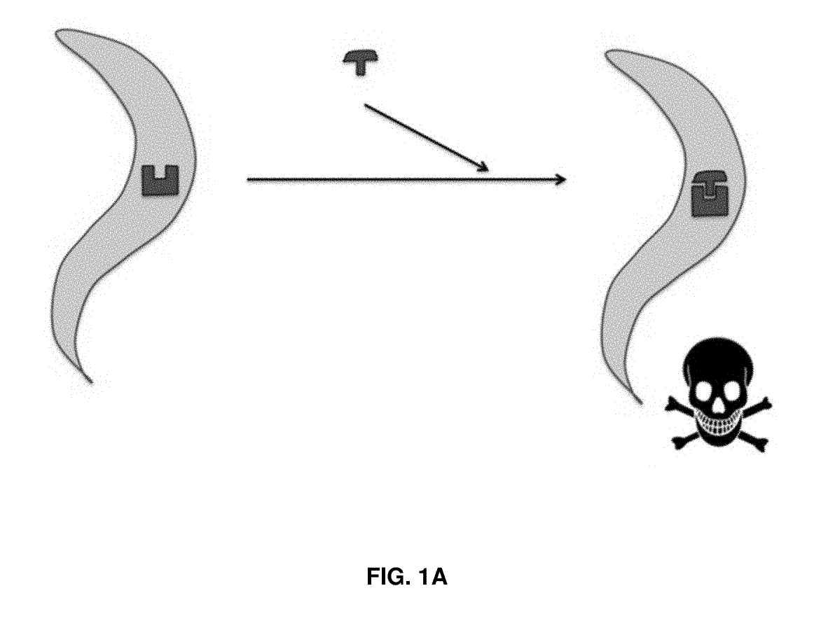

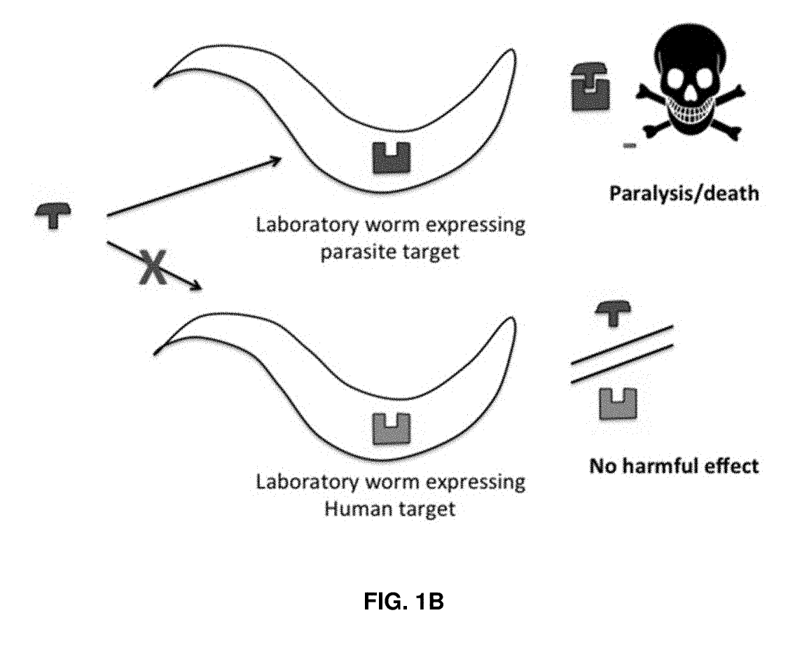

In some embodiments, provided is a screening method based on expressing parasitic nematode drug targets in the free-living nematode Caenorhabditis elegans, and testing compounds for the ability to cause paralysis and eventual death by activating those targets. (FIGS. 1A-1B.) This method maximizes screening efficiency by using C. elegans, which is easy to grow in the lab, while ensuring the compounds will specifically affect parasitic nematode targets. The method may further involve structurally modifying high-activity candidates identified in the screen to improve their activity profiles.

Compounds, such as those having structural similarity to 5-HT and related monoamines, can be screened accordingly. These compounds can be tested for paralytic activity using four worm strains: native C. elegans, C. elegans lacking all 5-HT receptors, C. elegans expressing the human 5-HT_{1A} receptor, and C. elegans expressing Mi-ser-4. A molecule that preferentially paralyzes the Mi-ser-4 expressing worms (i.e., it can penetrate the worm cuticle and selectively activate the M. incognita receptor, but not the C. elegans or human receptors, or other targets unrelated to the 5-HT system) is an ideal hit in the assay. Positive compounds can be tested in electrophysiological assays to confirm their activity at the M. incognita receptors. Further, positive compounds can be tested for paralytic activity toward M. incognita J2 larvae, and for the ability to protect host plants from M. incognita infestation.

In one non-limiting example, C. elegans strains expressing M. incognita SER-4, a validated drug target, can be generated, and expression can be confirmed using behavioral and electrophysiological assays. Paralysis activity is assayed for, and positive compounds are tested in electrophysiological assays to confirm their activity toward Mi-SER-4. M. incognita is closely related to C. elegans, so expression of M. incognita SER-4 in C. elegans is possible.

### EXAMPLES

### Example I—Heterologous Expression in Remodeled C. Elegans: A Platform for Monoaminergic Agonist Identification and Anthelmintic Screening

Monoamines, such as 5-HT and tyramine (TA), paralyze both free-living and parasitic nematodes when applied exogenously. Serotonergic agonists have been used to clear Haemonchus contortus infections in vivo. Since nematode cell lines are not available and animal screening options are limited, a screening platform has been developed to identify monoamine receptor agonists. Key receptors were expressed heterologously in chimeric, genetically-engineered Caenorhabditis elegans, at sites likely to yield robust phenotypes upon agonist stimulation. This approach preserves the unique pharmacologies of the receptors, while including nematode-specific accessory proteins and the nematode cuticle. Importantly, the sensitivity of monoamine-dependent paralysis can be increased dramatically by hypotonic incubation or the use of bus mutants with increased cuticular permeabilities. In this Example, it is demonstrated that the monoamine-dependent inhibition of key interneurons, cholinergic motor neurons, or body wall muscle inhibit locomotion and cause paralysis. Specifically, 5-HT paralyzed C. elegans 5-HT receptor null animals expressing either nematode, insect, or human orthologues of a key Gαₒ-coupled 5-HT₁-like receptor in the cholinergic motor neurons. Importantly, 8-OH-DPAT and PAPP, 5-HT receptor agonists, differentially paralyzed the transgenic animals, with 8-OH-DPAT paralyzing mutant animals expressing the human receptor at concentrations well below those affecting its C. elegans or insect orthologues. Similarly, 5-HT and TA paralyzed C. elegans 5-HT or TA receptor null animals, respectively, expressing either C. elegans or H. contortus 5-HT or TA-gated Cl⁻ channels in either C. elegans cholinergic motor neurons or body wall muscles. Together, these data indicate that this heterologous, ectopic expression screening approach is useful for the identification of agonists for key monoamine receptors from parasites, and can be used for the identification of ligands for a host of anthelmintic targets. Further, this Example demonstrates the usefulness of these transgenic C. elegans for agonist identification and anthelmintic screening.

A heterologous, ectopic over-expression approach has been developed to provide a nematode screening platform for selective agonist identification, exploiting the unique experimental advantages of the C. elegans model system. It has been demonstrated that exogenous monoamines, such as serotonin (5-HT), dopamine (DA), and tyramine (TA), each paralyze C. elegans and, where examined, parasitic nematodes. In each case, the key C. elegans receptors mediating this locomotory inhibition have been identified and functionally localized, with each operating at a different level within the locomotory circuit: 5-HT in a few key interneurons, including the two AIB interneurons, DA in the cholinergic motor neurons, and TA in head muscle and additional interneurons associated with locomotory decision-making Quintuple 5-HT receptor null C. elegans (5-HT quint) that do not express any previously identified 5-HT receptors and do not respond to exogenous 5-HT have been previously constructed, to identify essential roles for the Gαₒ-coupled 5-HT₁-like SER-4 and the unique 5-HT-gated Cl⁻ channel, MOD-1 in 5-HT-dependent locomotory paralysis. SER-4 agonists appear to function as anthelmintics in vivo and have been used to clear Haemonchus contortus infections from gerbils. In the present Example, SER-4 and MOD-1 orthologues from parasitic nematodes, insects, and humans were ectopically expressed in either the cholinergic motor neurons or body wall muscles of quintuple C. elegans 5-HT receptor null animals that lack all known C. elegans 5-HT receptors. Agonist-dependent receptor activation at these sites causes robust phenotypes that can be readily adapted for agonist screening. For example, the activation of a ligand-gated Cl⁻ channel in body wall muscles hyperpolarize the muscle and significantly inhibit locomotion, while the activation of a Cl⁻ channel or Gαₒ-coupled GPCR on the cholinergic motor neurons significantly inhibit acetylcholine (ACh) release from the motor neurons and inhibit both muscle contraction and thus locomotion.

Materials and Methods

Strains and Reagents

bus-8 (e2968), bus-16 (e2802), and bus-17 (e2800) were obtained from the Caenorhabditis Genetics Center (CGC). ser-5 (tm2654);ser-4 (ok512);mod-1 (ok103);ser-7 (tm1325) ser-1 (ok345) (5-HT quint), ser-5 (tm2654);mod-1 (ok103);ser-7 (tm1325) ser-1 (ok345) (SER-4 quad), and lgc-55 (tm2913);tyra-3 (ok325) tyra-2 (tm1846) ser-2 (pk1357) (TA quad) were generated as described previously. All strains were maintained on NGM plates with OP50 at 16° C. The cDNA clone of Drosophila melanogaster 5-HT_{1A} (RE57708) was ordered from the Drosophila Genomics Resource Center (DGRC), the cDNA clone Human HTR1A (MGC: 167873; clone ID: 9020250) from GE Healthcare Dharmacon Inc., and cDNA clones of Haemonchus contortus (Hco) lgc-55 and mod-1 orthologues were kindly provided by Dr. Sean Forrester. The unc-17β promoter, RM#621p, was obtained from Dr. James Rand. The integrated AIB::HisCl1 in N2 (cx15457) animals were a kind gift from Dr. Cornelia Bargmann.

Serotonin (5-HT) (H7752-25G), tyramine (TA) (T2879-25G), 8-OH DPAT (H141-25MG), sumatriptan succinate (S1198-10MG), PAPP (S009-25MG), and histamine dihydrochloride (H7250-5G) were purchased from Sigma Life Sciences. Stock solutions (50 mM) of 5-HT, TA, 8-OH-DPAT, sumatriptan, and histamine were made up in distilled water, PAPP in 100% ethanol. The constituent for nematode growth media (NGM), potassium phosphate monobasic (KH₂PO₄; P285-3), sodium chloride (NaCl; S271-3), calcium chloride dehydrate (CaCl₂.2H₂O; C79-500), magnesium sulfate heptahydrate (MgSO₄.7H₂O; BP213-1), tryptone (BP1421-2), and agar (DF0812071) were purchased from Thermo Fisher Scientific Inc., and cholesterol (C3045-5G) was purchased from Sigma Life Science.

Fusion PCR and Transgenic Lines

All transgenic constructs were created by overlap fusion PCR. All transgenes contain a GFP marker (with unc-54 30-UTR) at the 3′-end. PCR products from multiple reactions were pooled and co-injected with coelomocyte-RFP screening marker into the appropriate null backgrounds. Once generated, transgenic animals were frozen in liquid nitrogen and thawed fresh weekly for assay. Multiple transgenic lines from each construct were examined.

Paralysis Assay

Fresh agar plates (without NaCl, KH₂PO₄, MgSO₄, CaCl₂, tryptone, and cholesterol) containing 5-HT, TA, PAPP, sumatriptan, or 8-OH DPAT at desired concentrations were made daily. For assays involving bus mutants, fresh NGM agar plates (with NaCl, KH₂PO₄, MgSO₄, CaCl₂, tryptone, and cholesterol) containing 5-HT were used for all assays. For assays with AIB::HisCl1 (cx15457) animals, freshly poured NGM agar or agar only plates containing 10 mM and 2 mM histamine were used. NGM agar plates were prepared as previously described.

For all paralysis assays, well-fed, transgenic young adults expressing RFP screening markers were picked 2 hrs prior to assay and maintained on NGM plates with E. coli OP50. For the assay, 10 animals were transferred to assay plates (agar only for all assays and NGM agar for assays with bus mutants) containing the appropriate drug, and motility was assessed at intervals of 5 min for 30 min. Experiments with sumatriptan were carried out for 60 min, with motility assessed every 5 min. All assays were conducted in the absence of food, i.e., OP50. Animals that moved less than 1 body bend/20 s were counted as paralyzed. Each transgenic line was assayed at least 3 times with 10 animals/assay for each agonist concentration. Data is presented as % paralyzed±SE over drug exposure time (min). Dose-response curves and EC₅₀s were then generated using a variable slope nonlinear regression model with GraphPad Prism 6 software. Drug concentrations were log 10-transformed prior to analysis.

Accession Numbers

The accession numbers of the proteins involved in this Example are C. elegans SER-4 (accession no. NP_497452), C. elegans LGC-55 (accession no. NP_507870), C. elegans MOD-1 (accession no. CCD72364), D. melanogaster 5-HT_{1A} (accession no. NM_166322.2), D. melanogaster HisCl1 (accession no. Q9VGI0), human HTR1A (accession no. BC136263), H. contortus LGC-55 (accession no. ACZ57924.1), and H. contortus MOD-1 (accession no. ADM53350.1).

Results

Rationale

The monoamines 5-HT, DA, and TA each dramatically inhibit locomotion in C. elegans when applied exogenously at concentrations high enough to overcome the permeability barrier of the nematode cuticle, ultimately resulting in paralysis. Using the C. elegans model, the receptors involved in monoamine-dependent locomotory inhibition have been identified and localized. The key receptors involved in 5-HT, DA, and TA inhibition each function at a different level in the locomotory circuit with 5-HT-dependent paralysis requiring the expression of the Gαo-coupled, 5-HT₁-like receptor, SER-4, and the 5-HT-gated Cl⁻ channel, MOD-1, in a limited number of interneurons, including the two AIBs. Unfortunately, since nematode cell lines are not available and the maintenance of parasitic nematodes outside their hosts is problematic, screening platforms for anti-nematodal activity have been limited and do not usually incorporate the nematode cuticle or potentially important nematode accessory proteins.

The present Example develops a screening platform for nematode monoamine receptor agonists in “chimeric” genetically-engineered C. elegans by heterologously expressing 5-HT and TA receptors at sites likely to yield robust phenotypes upon agonist stimulation. Previously, a range of behaviors in C. elegans null animals have been rescued with the expression of proteins from the parasites, validating this approach. In this Example, locomotion was examined as an endpoint for heterologous, ectopic expression, as the neurons and circuits modulating locomotion in C. elegans and parasitic nematodes are conserved, can be readily assessed by established screening assays, and have always been the primary target for the majority of existing anthelmintics. Specifically, the following were expressed: 1) Gαₒ-coupled, 5-HT₁-like receptors, or 5-HT/TA-gated Cl⁻ channels in the cholinergic motor neurons of C. elegans mutants lacking any 5-HT or TA receptors, respectively, because robust agonist-dependent Gαₒ signaling or hyperpolarization, respectively, would dramatically inhibit ACh release and locomotion, and 2) 5-HT or TA-gated Cl- channels in body muscle of C. elegans mutants lacking any 5-HT or TA receptors, respectively, because agonist-dependent muscle hyperpolarization would cause paralysis.

5-HT Inhibits Locomotion in 5-HT Receptor Null Animals Expressing 5-HT₁-Like Receptors in the AIB Interneurons or Cholinergic Motor Neurons

The role of the C. elegans 5-HT₁-like receptor SER-4 in 5-HT-dependent paralysis is well documented. The utility of the H. contortus SER-4 orthologue, 5-HT₁HC, as an anthelmintic target has been validated previously both in vivo and in vitro. Locomotion in C. elegans has been assessed previously using a number of different assays, many of which can be readily adapted for screening. For example, automated thrashing assays allow thousands of compounds to be easily screened per day. Monoamine-dependent locomotory inhibition and paralysis has been quantified on agar plates (sinusoidal body bends) and in liquid medium (C-shaped “swimming”), containing either M9 buffer or water. The permeability of the C. elegans cuticle varies depending on incubation conditions, with much less 5-HT required in water than in salt-containing media (M9). Without wishing to be bound by theory, it is believed this is because of an increased cuticular permeability under hypotonic conditions.

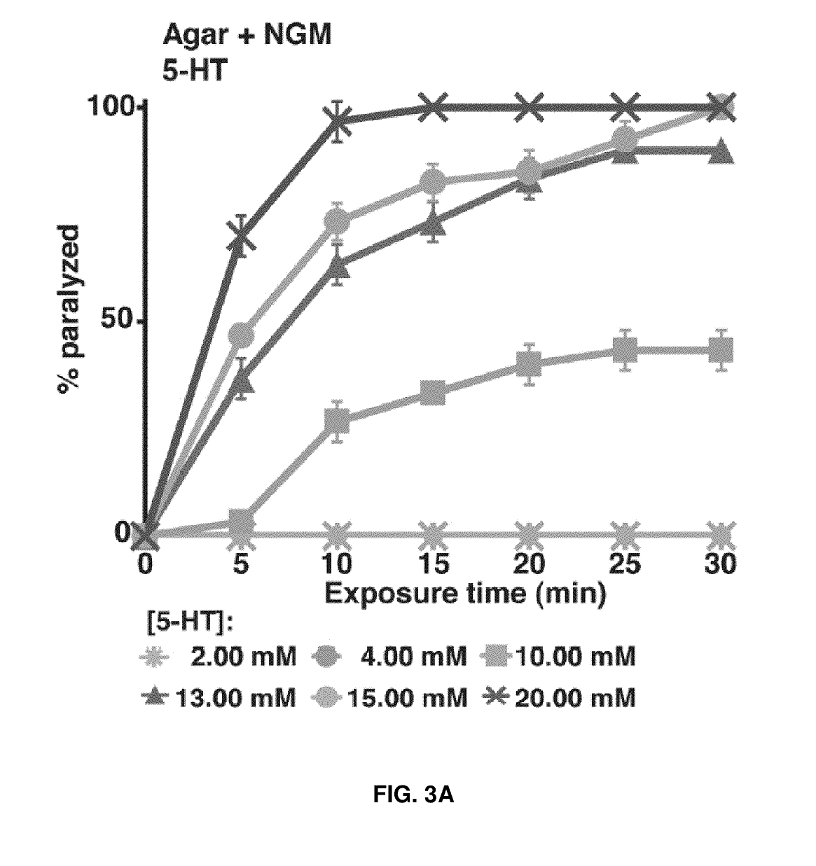

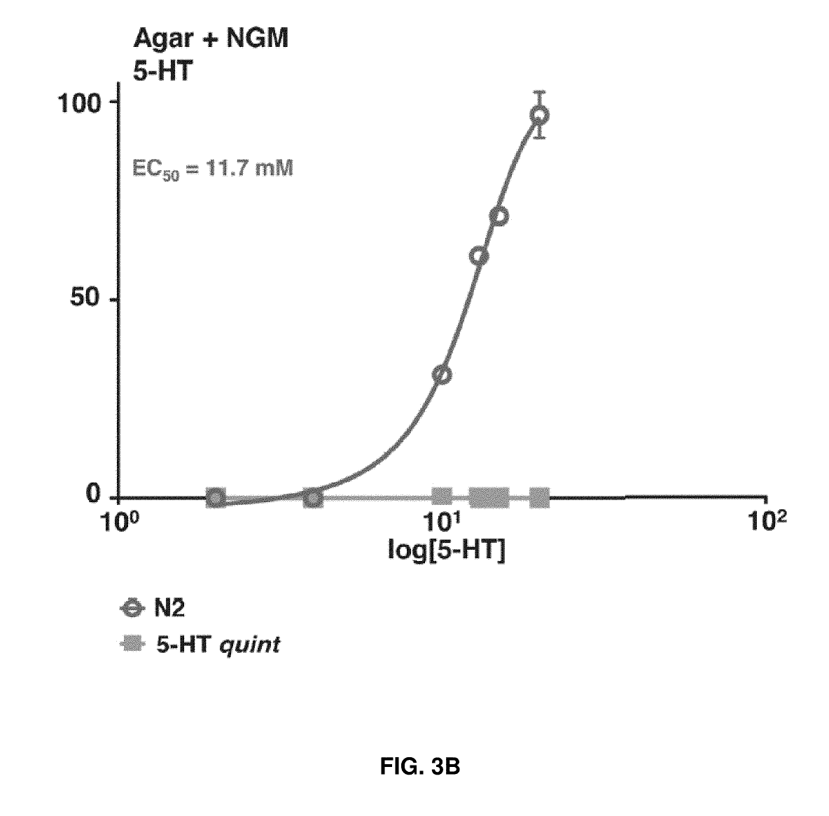

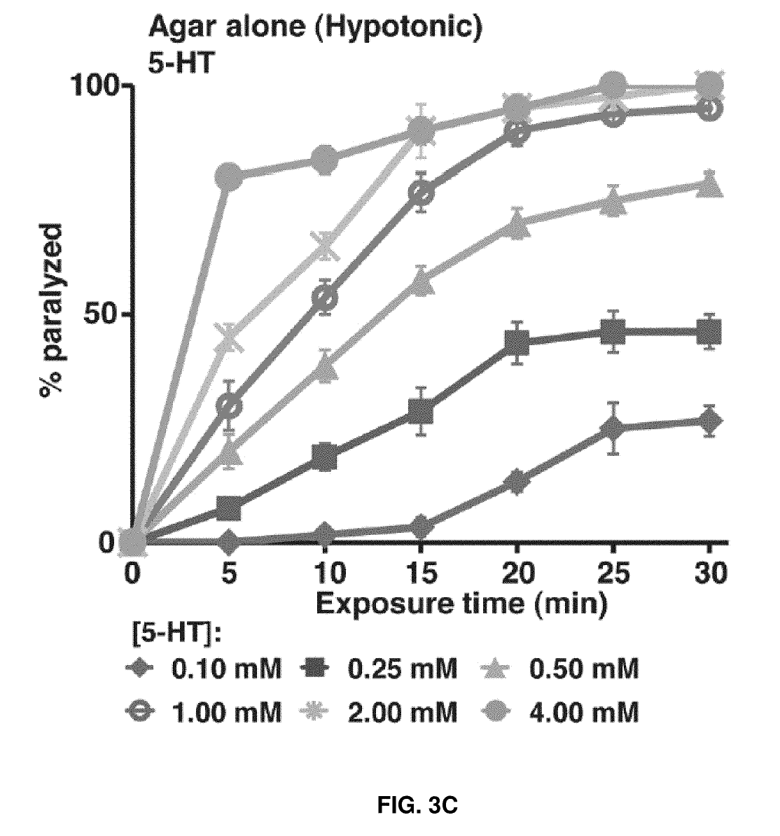

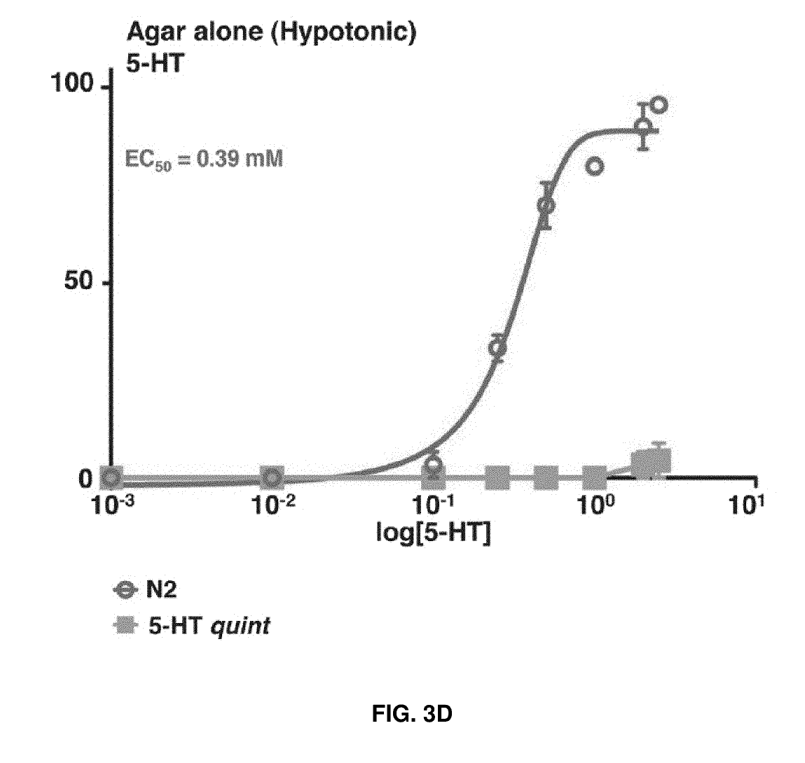

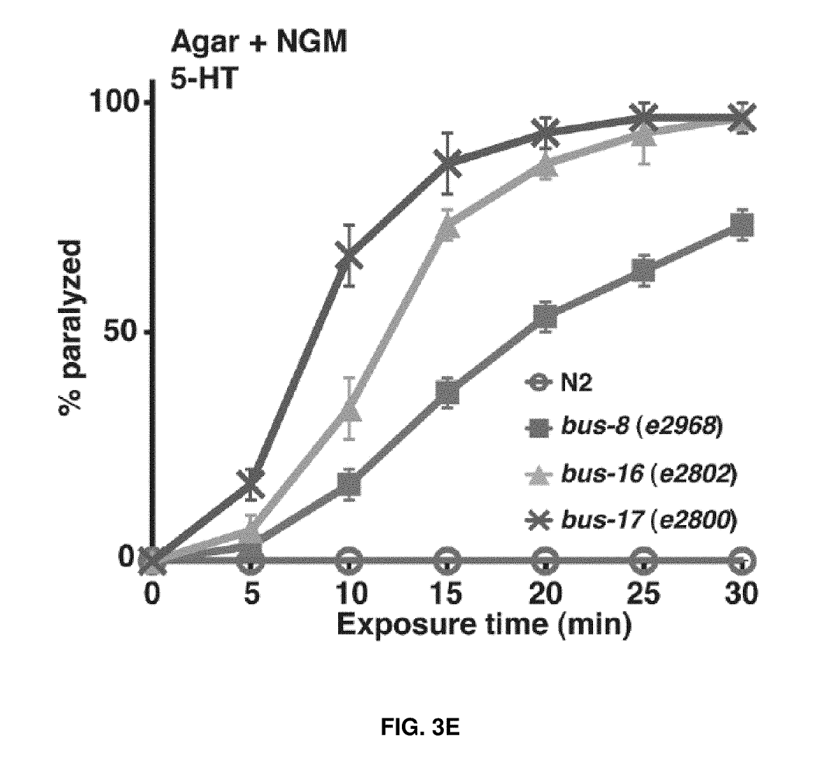

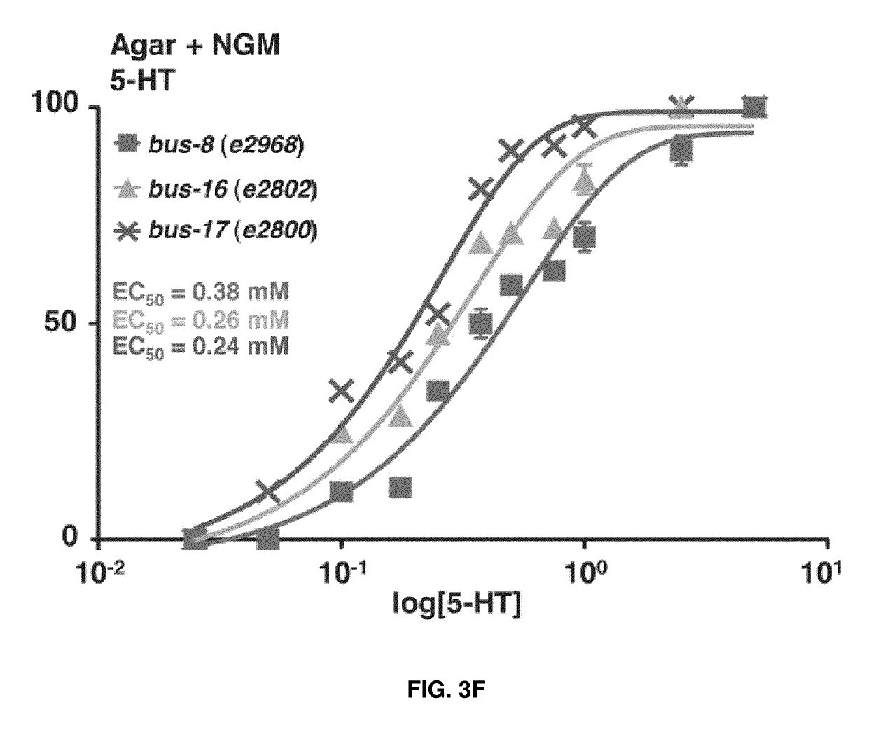

Previously, locomotion under standard C. elegans culture conditions was assayed on NGM agar plates. Under these conditions, 15 mM 5-HT initiated a rapid paralysis in wild type animals, and ser-5;mod-1;ser-7 ser-1 quadruple null (SER-4 quad) animals. 5-HT had no effect on locomotion in 5-HT quint animals that lack all previously identified 5-HT receptors (FIGS. 3A-3B). This 5-HT-dependent paralysis was not the classical spastic paralysis associated with cholinergic agonists, such as levamisole, or the flaccid paralysis associated with glutamatergic agonists, such as ivermectin, but instead resulted more from “locomotory confusion,” with animals unable to effectively integrate conflicting sensory inputs to initiate and sustain forward/backward locomotion. The C. elegans cuticle is more impermeable than those of some of the parasitic nematodes. Therefore, since the concentration of 5-HT required for maximal paralysis was quite high (15 mM) in these short term assays, presumably to overcome cuticular permeability, these animals were re-assayed under hypotonic conditions on agar plates without salt (non-NGM) (FIGS. 3C-3D). Attempts at achieving 5-HT paralysis in water were unsuccessful, as a majority of the animals burst soon (within 5 min) after exposure to water. However, in a hypotonic environment (agar alone without NGM), much lower concentrations of 5-HT were required for inhibition of wild type animals, with 1 mM 5-HT yielding 50% paralysis after 10 min exposure (EC50 about 0.4 mM) (FIGS. 3C-3D).

In addition to hypotonic incubation, 5-HT-dependent paralysis was also examined in a number of C. elegans mutants that exhibit increased cuticular permeability. For example, a series of bus mutants that exhibit increased cuticular permeability that have been hypothesized to be excellent vehicles for small molecule screening have been previously identified. As noted in FIGS. 3E-3F, many of the bus mutants are hypersensitive to 5-HT dependent paralysis, even under isotonic assay conditions (on NGM agar plates). For example, bus-17 mutants are acutely paralyzed after 10 min on 5-HT with an EC₅₀ of about 0.24 mM, which is substantially lower than that observed in wild-type animals incubated under the same conditions (EC₅₀=11.5 mM) (FIG. 1F). These results indicate that these mutants are useful for agonist identification, especially when only limited amounts of compound are available. Indeed, it is possible to select mutants that exhibit cuticular permeabilities that mimic those of individual parasites. Unfortunately, these mutants are also sensitive to hypotonicity and burst under the hypotonic conditions used in the present Example, so they could not be used in combination with hypotonicity to further increase sensitivity. Therefore, unless specified, hypotonic conditions were used to assay the transgenic animals described below.

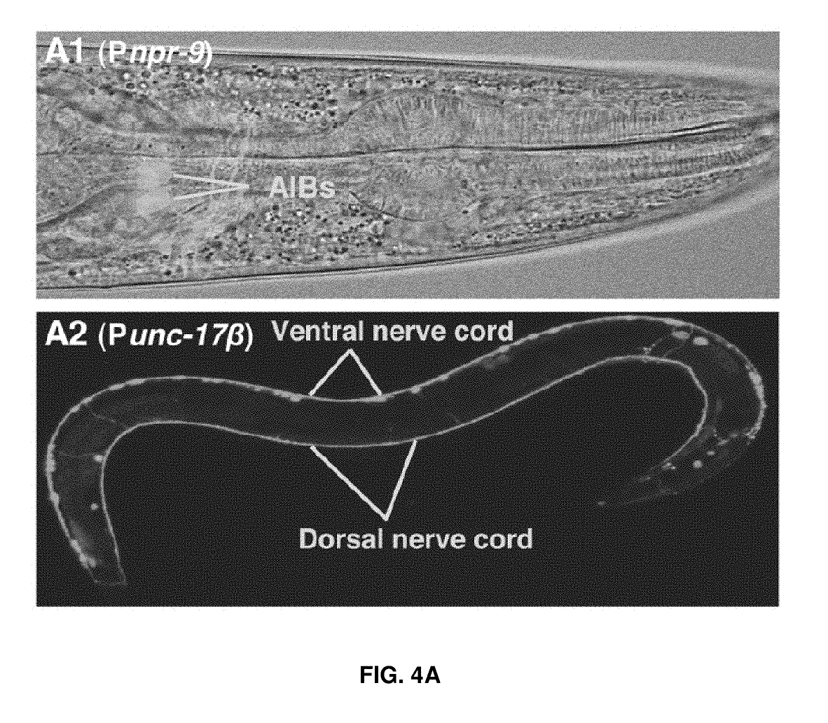

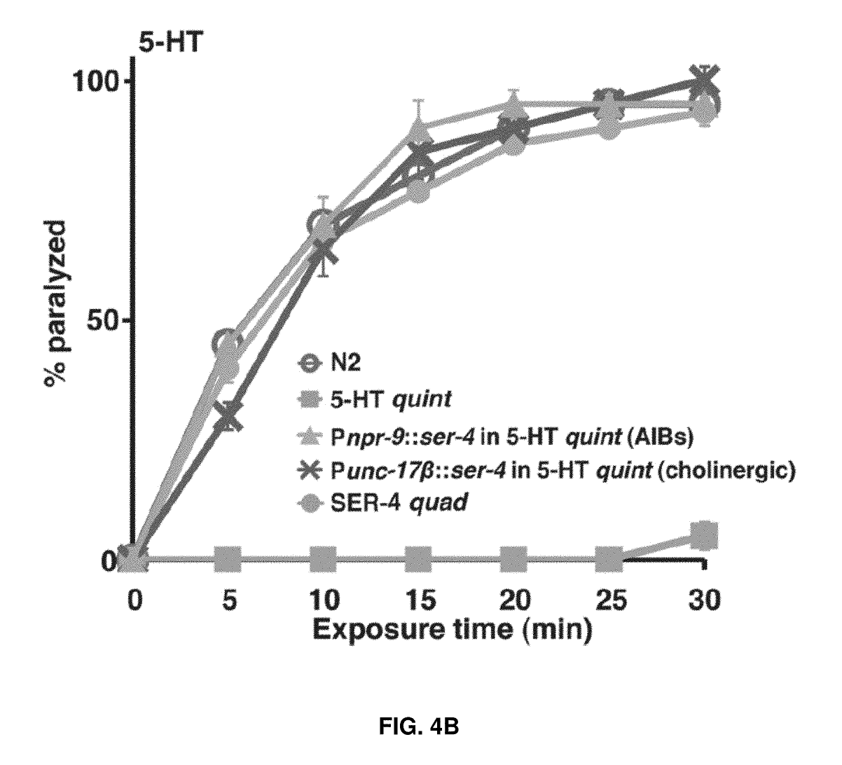

A ser-4::gfp transgene is expressed in a limited number of neurons, including the AIBs. Therefore, SER-4::GFP was specifically expressed in either the AIB interneurons (Pnpr-9) or ectopically, in the cholinergic motor neurons (Punc-17β) of the 5-HT quint. Expression was confirmed by GFP fluorescence (FIG. 4A). 5-HT quint animals expressing SER-4 in either the AIBs or cholinergic motor neurons were rapidly paralyzed by 5-HT (FIG. 4B). On 5-HT, although 5-HT quint animals expressing SER-4 in the AIBs alone moved only infrequently, they initiated backward locomotion for a short distance when prodded with a blunt platinum wire at the tail, indicating that they were probably unable to process conflicting locomotory signals. In contrast, animals expressing SER-4 in the cholinergic motor neurons were fully paralyzed and did not move when prodded.

Use of Heterologous Expression for Agonist Identification

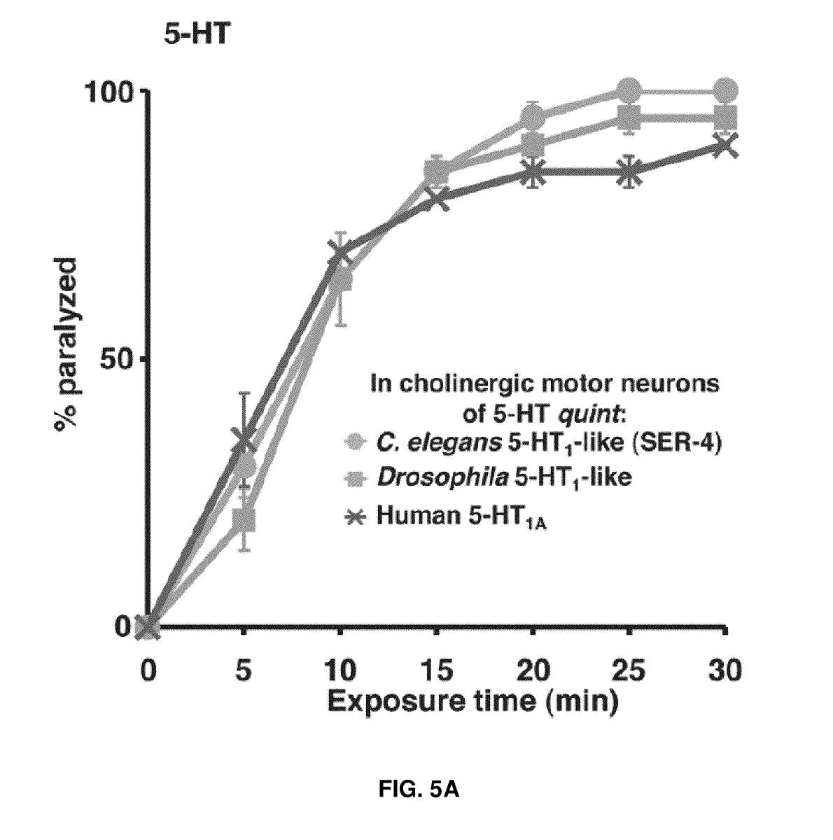

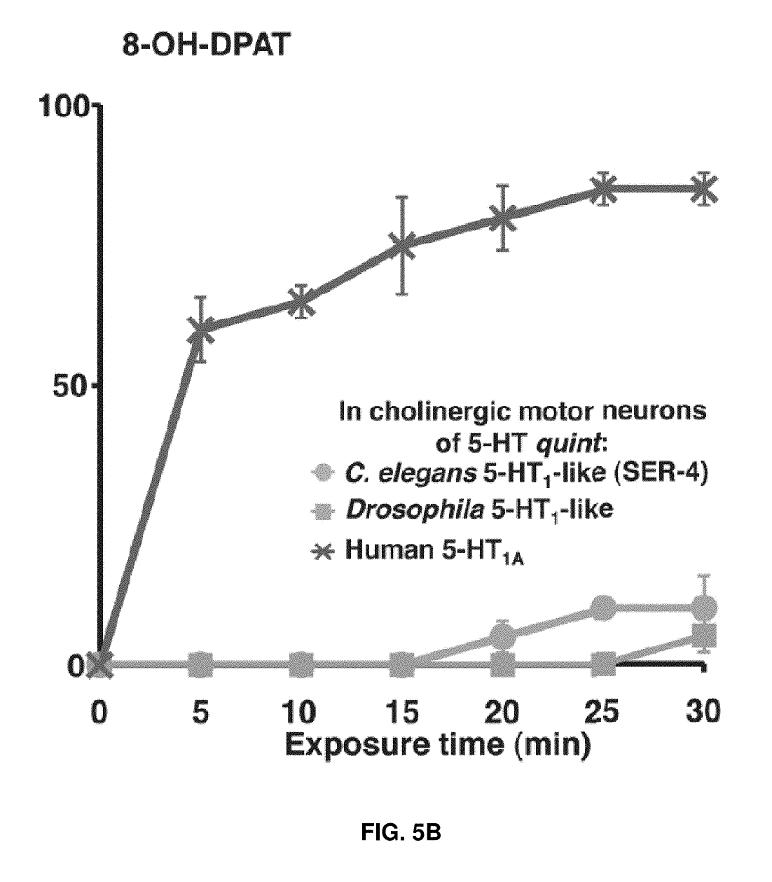

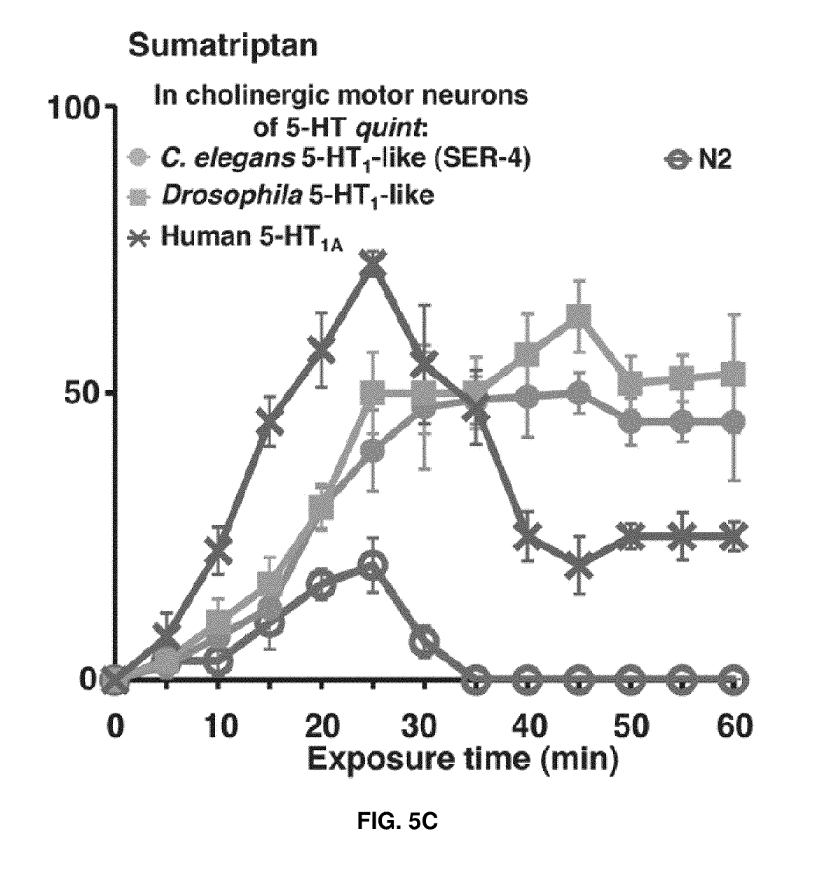

To demonstrate the utility of this screening approach, the Drosophila 5-HT₁ orthologue (5HT_{1A}) or the human 5-HT-1A receptor (HTR_{1A}) were also expressed specifically in the cholinergic motor neurons (Punc-17β) of 5-HT quint animals Locomotion in animals from both transgenic lines was dramatically inhibited by exogenous 5-HT, demonstrating that the receptors were functionally expressed (FIG. 5A). To demonstrate the specificity of these chimeric C. elegans for agonist identification, the effect of 8-hydroxy-2-(di-n-propylamino) tetralin (8-OH-DPAT), a subtype-selective agonist for the human 5-HT_{1A} receptor, sumatriptan succinate, a selective mammalian 5-HT1B/D agonist, and p-amino-phenethyl-m-trifluoromethylphenyl piperazine (PAPP) were examined 8-OH-DPAT rapidly paralyzed the 5-HT quint animals expressing the human 5-HT_{1A} receptor (FIG. 5B). In contrast, 8-OH-DPAT, even at 2 mM, had no effect on locomotion in 5-HT quint animals expressing either Drosophila or C. elegans 5-HT₁ receptor orthologues, indicating the conservation of ligand receptor specificity in chimeric C. elegans (FIG. 5B). Sumatriptan, at low concentrations, is a selective mammalian 5-HT1B/D agonist, and in the present Example, sumatriptan was much less effective than 8-OH-DPAT in initiating paralysis. For example, 0.5 mM sumatriptan had no effect on locomotion in either wild type or transgenic animals expressing 5-HT_{1A} receptor orthologues in cholinergic motor neurons and, even at higher concentrations, failed to fully paralyze animals expressing the human 5-HT_{1A} receptor. In addition, although animals expressing the human 5-HT_{1A} receptor responded to increased sumatriptan concentrations more rapidly, these locomotory effects were transient and reduced dramatically after 25 min, likely due to receptor desensitization (FIG. 5C). In contrast, paralysis increased with prolonged sumatriptan exposure in animals expressing either the C. elegans or Drosophila receptors, demonstrating kinetic differences between the orthologous receptors.

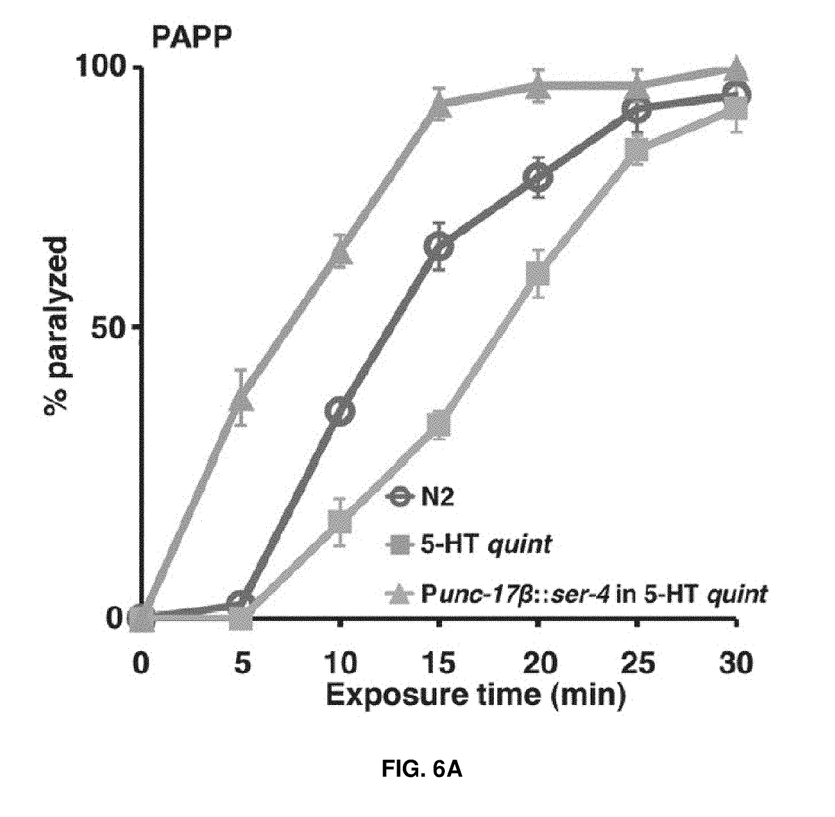

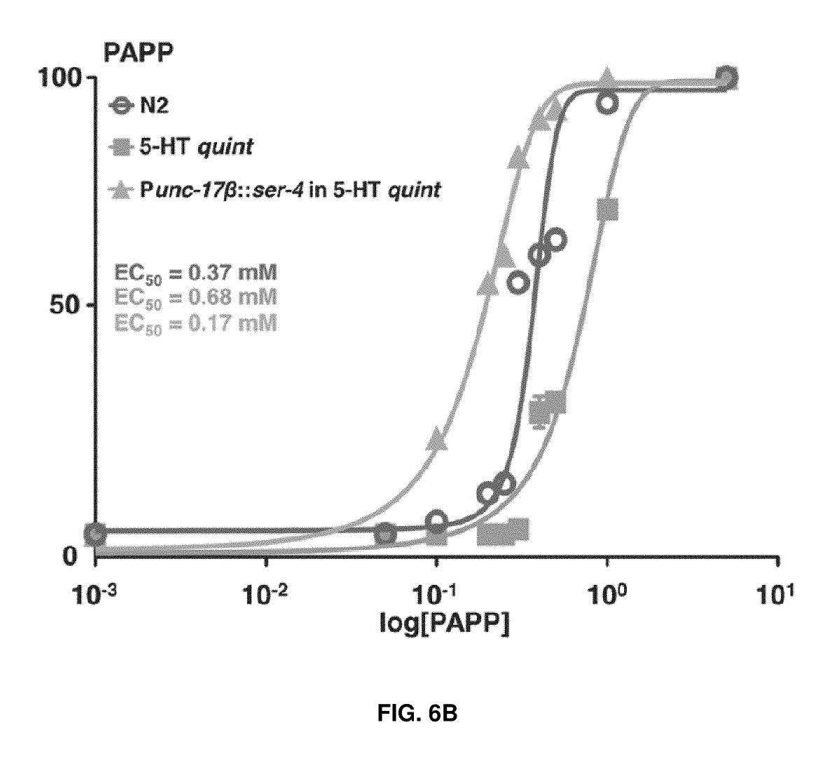

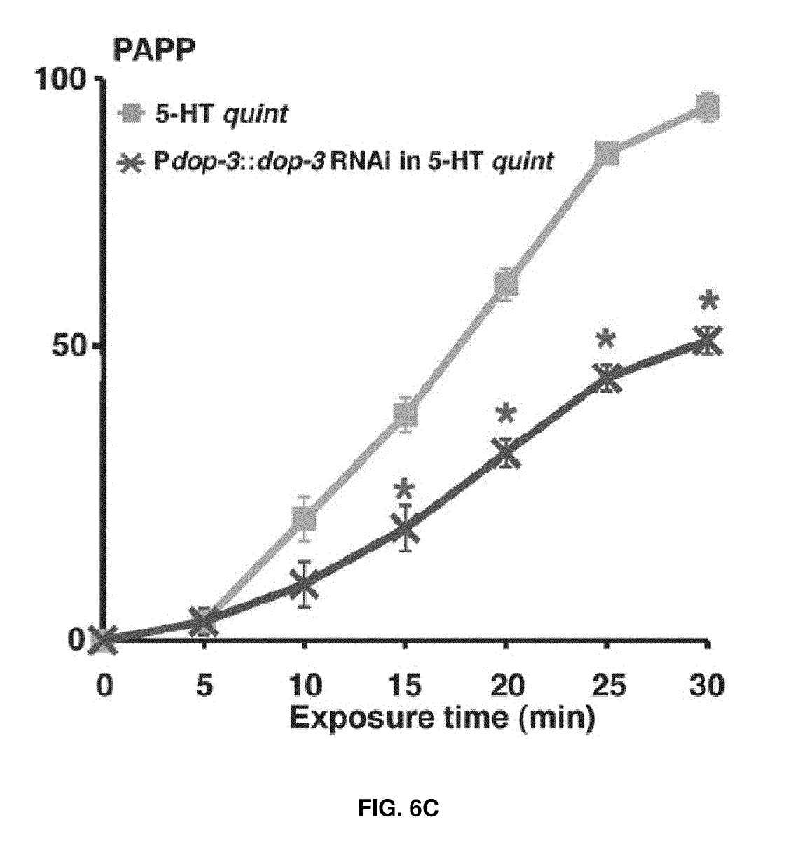

PAPP, a high affinity agonist for the H. contortus 5-HT₁-like receptor, paralyzes H. contortus L3s in vitro and clears experimental H. contortus infections from gerbils. PAPP initiated a rapid paralysis in wild type animals (EC₅₀=0.37 mM) and, even more rapidly, in 5-HT quint animals expressing the C. elegans SER-4 in the cholinergic motor neurons (EC₅₀=0.17 mM), supporting the identification of PAPP as a 5-HT₁-like receptor agonist (FIGS. 6A-6B). In contrast, and somewhat surprisingly, at higher concentrations (≧0.5 mM), PAPP also paralyzed 5-HT quint animals (EC₅₀=0.68 mM) that were unaffected by 5-HT, indicating that, in addition to acting as a 5-HT₁-like receptor (SER-4) agonist, PAPP also acted at second target(s) (FIGS. 6A-6B). Since exogenous TA and DA also paralyze C. elegans, it is now believed that, at higher concentrations, PAPP is activating additional monoamine receptors. DA-dependent paralysis requires the expression of the Gαₒ-coupled DA receptor DOP-3 in the cholinergic motor neurons. Therefore, dop-3 expression was knocked down in the 5-HT quint animals using dop-3 RNAi driven by the dop-3 promoter. As noted in FIG. 6C, dop-3 RNAi knockdown in this background significantly reduced PAPP-dependent paralysis, indicating that DOP-3 is a secondary PAPP target. Screening was conducted to identify additional targets. Together, these data highlight the utility of this approach in preliminary drug screening, and demonstrate that it is useful for the identification of nematode-specific agonists.

The Activation of Monoamine-Gated Cl⁻ Channels in Cholinergic Motor Neurons or Body Wall Muscles Causes Locomotory Paralysis

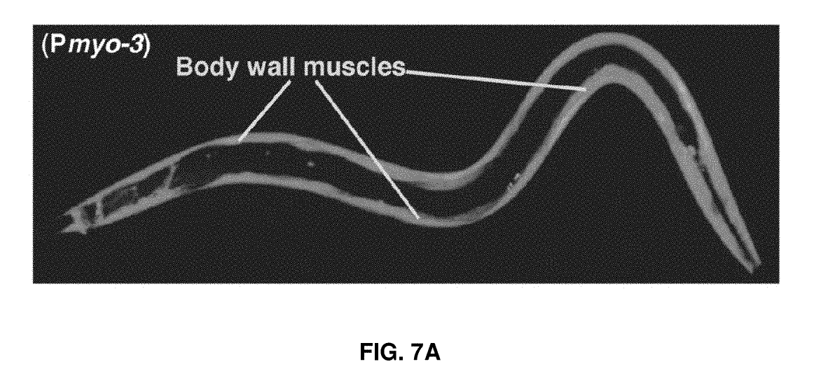

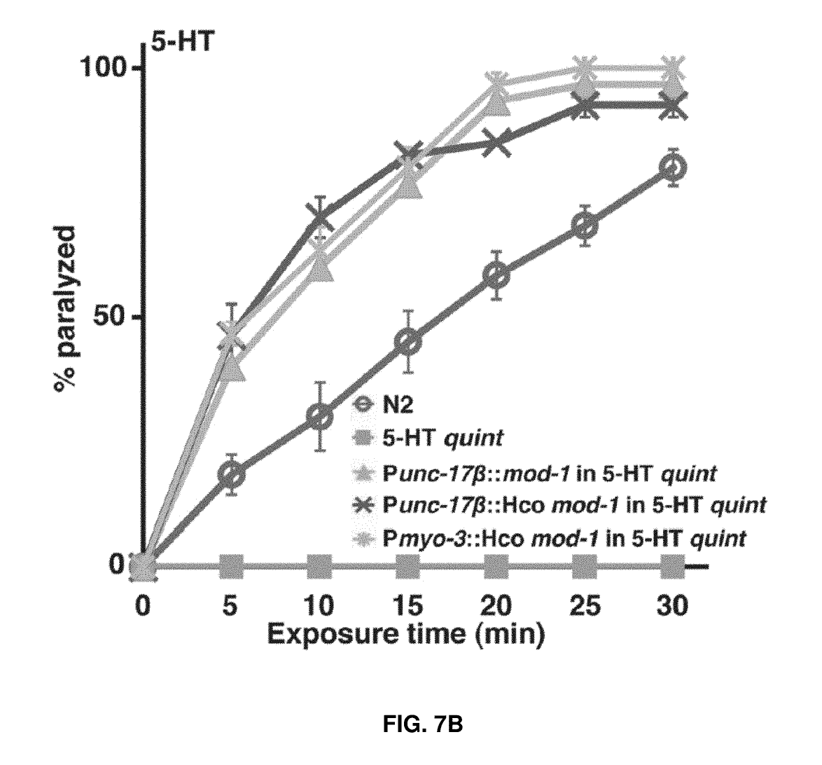

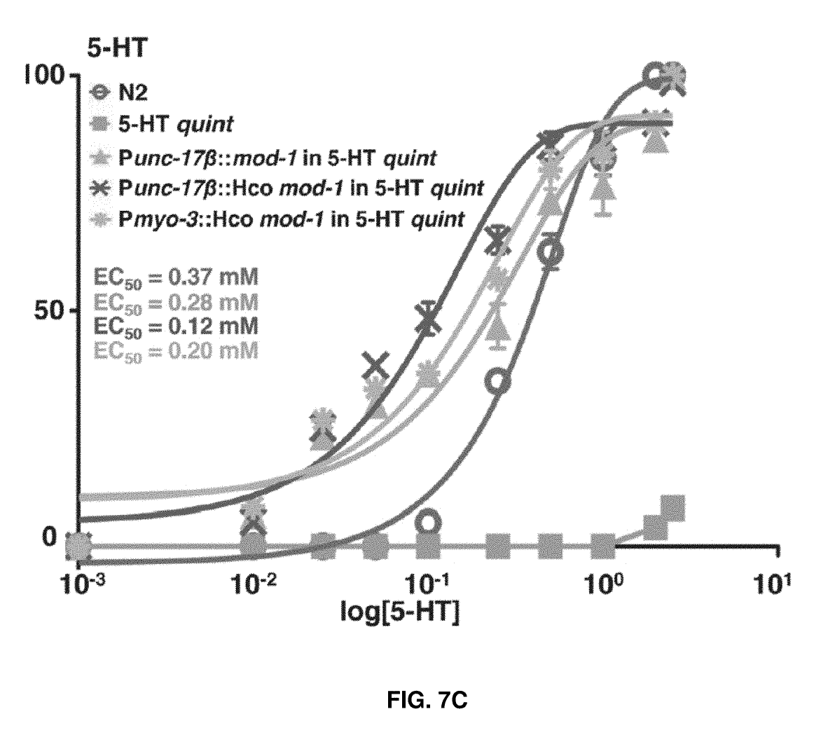

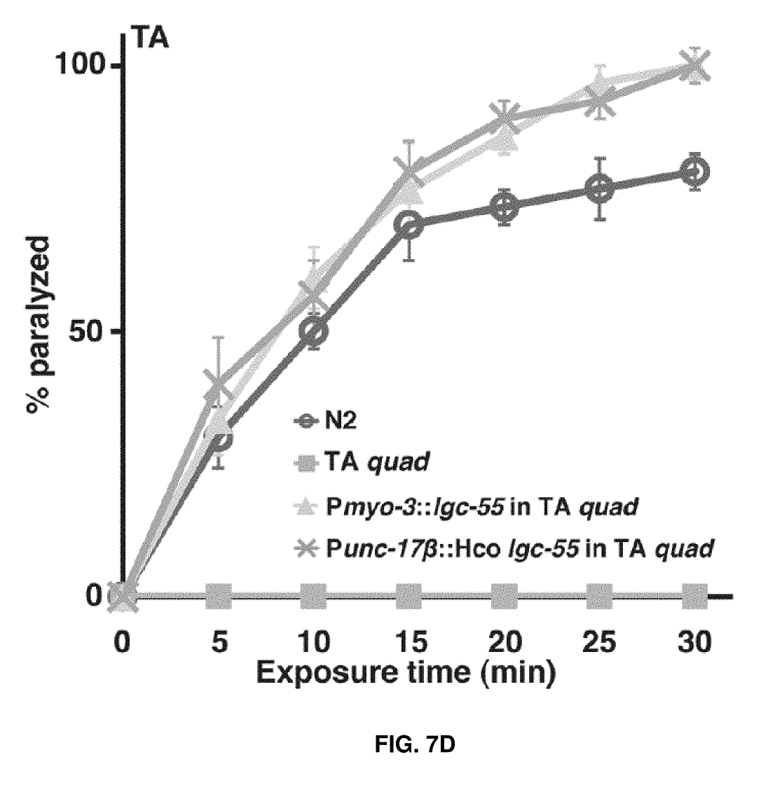

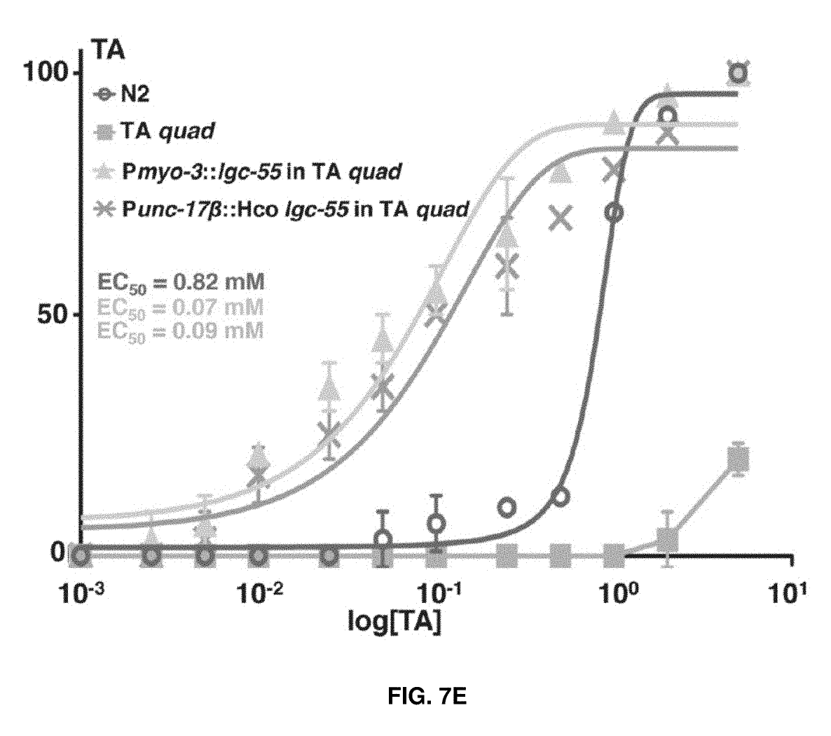

Nematodes also express a unique family of monoamine-gated Cl⁻ channels that appear to be highly conserved within the phylum, including the C. elegans 5-HT- and TA-gated Cl⁻ channels, MOD-1, and LGC-55, that play key roles in 5-HT- and TA-dependent muscle paralysis, respectively. The C. elegans MOD-1 and its H. contortus orthologue were expressed directly in either cholinergic motor neurons (Punc-17β) or body wall muscles (Pmyo-3) of 5-HT quint animals and 5-HT-dependent paralysis was assayed as described above. Muscle expression was confirmed by GFP fluorescence (FIG. 7A). As previously noted, 5-HT had no effect on locomotion in 5-HT quint animals, but rapidly paralyzed the 5-HT quint animals expressing either the C. elegans MOD-1 in the cholinergic motor neurons or the H. contortus (Hco) MOD-1 orthologue in cholinergic motor neurons or body wall muscle, with EC₅₀s of about 0.3 mM, 0.2 mM, and 0.2 mM, respectively (FIGS. 7B-7C). 5-HT-dependent paralysis was more rapid in the transgenic animals expressing MOD-1 orthologues in the cholinergic motor neurons than in wild type animals.

Similarly, LGC-55 was expressed in the body wall muscles (Pmyo-3) or its H. contortus orthologue in the cholinergic motor neurons (Punc-17β) of lgc-55;tyra-3 tyra-2 ser-2 quadruple TA receptor null (TA quad) animals TA quad animals lack all previously identified TA receptors and fail to respond to TA in a range of behavioral assays, including locomotion. TA had no effect on locomotion in the TA quad animals, but significantly inhibited locomotion in TA quad animals expressing either C. elegans LGC-55 in body wall muscles or H. contortus (Hco) LGC-55 orthologue in cholinergic motor neurons, each with EC₅₀ of about 0.1 mM (FIGS. 7D-7E). Together, these data indicate that monoaminergic activation of these Cl⁻ channels hyperpolarizes either the cholinergic motor neurons or body wall muscles and inhibits muscle contraction, as well as highlight the utility of chimeric C. elegans as a functional expression platform to identify ligand-gated Cl⁻ channels agonists for use as anthelmintics.

The Inhibition of AIB Signaling Causes “Locomotory Confusion” and Paralysis

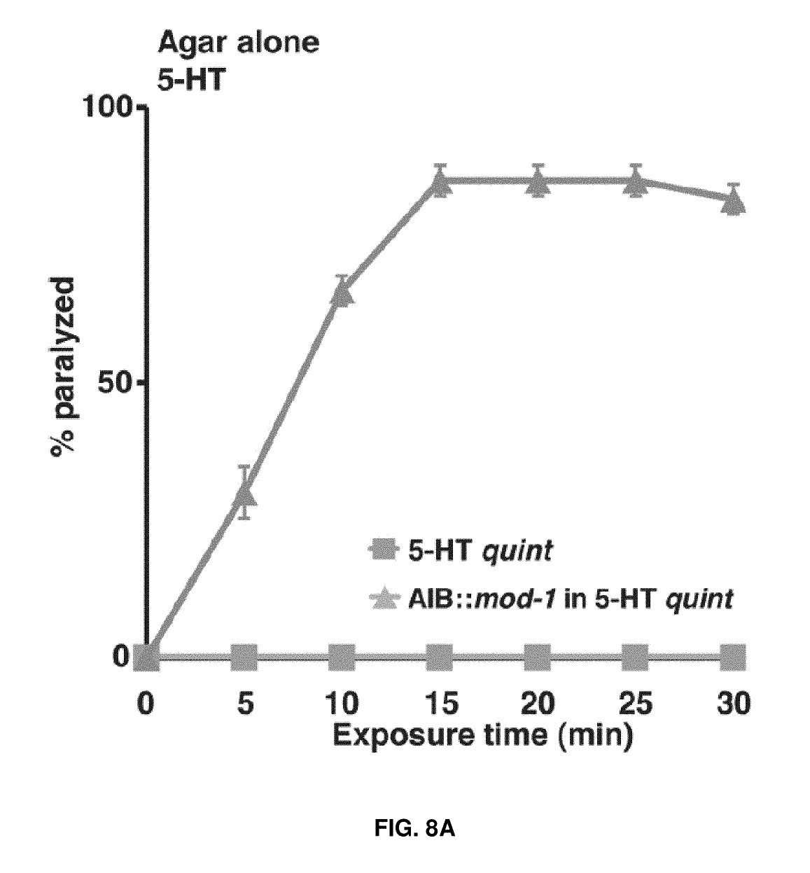

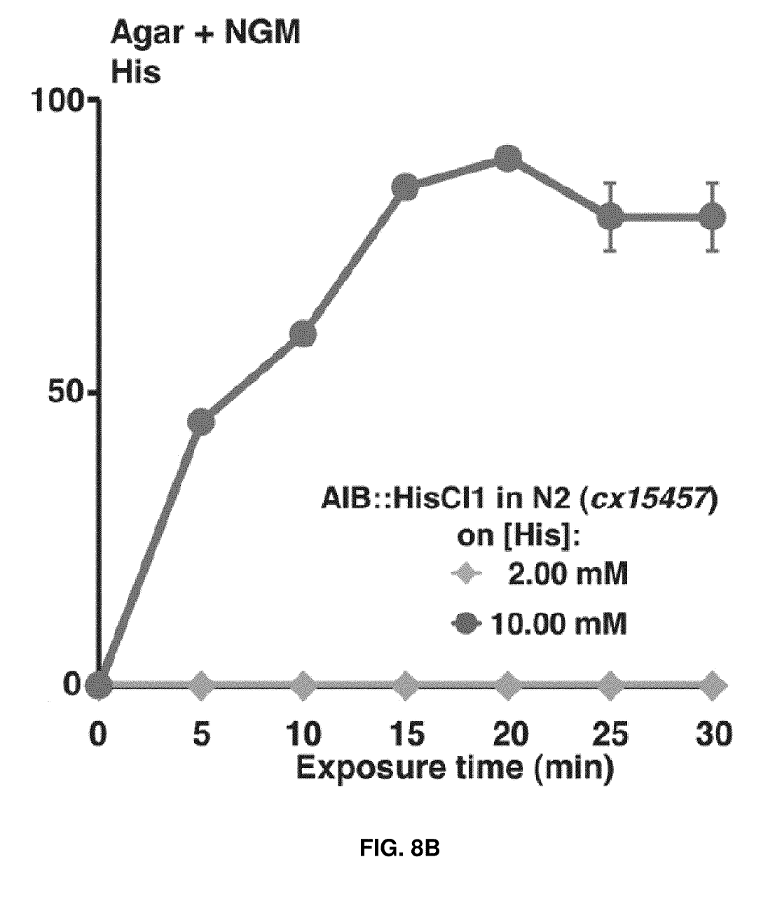

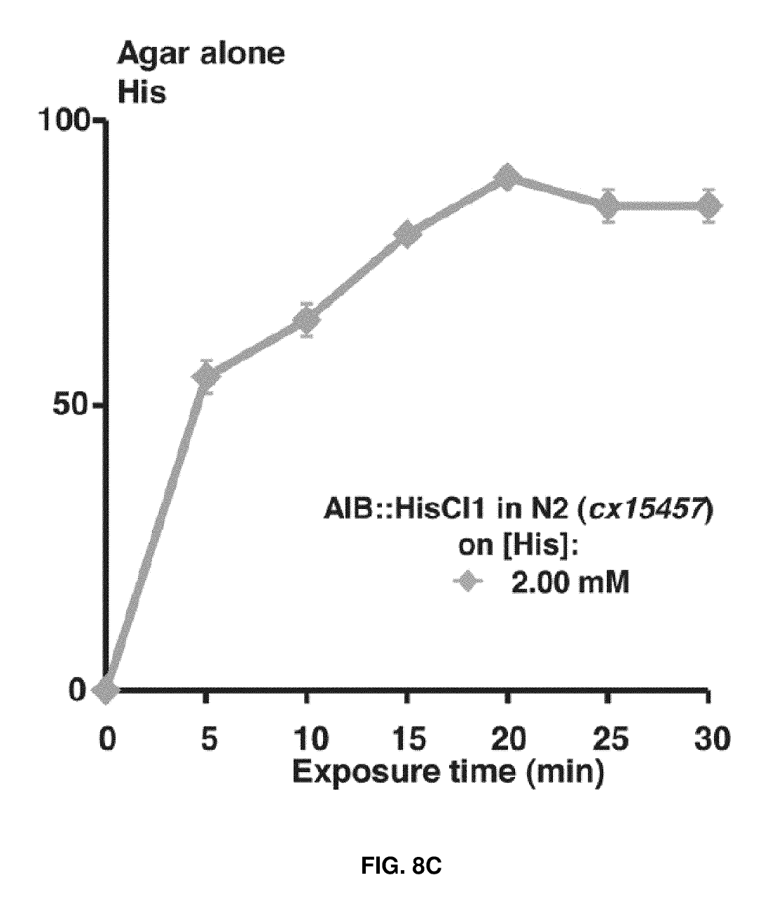

The results indicate that inhibiting AIB signaling by the expression of a Gαₒ-coupled 5-HT receptor in the AIBs of the 5-HT quint can cause paralysis (FIG. 4B). Similarly, the AIB-specific expression (Pinx-1) of the 5-HT-gated Cl⁻ channel, MOD-1 can also cause paralysis (FIG. 8A). In contrast, ablation of the AIBs does not cause paralysis. The activation of a Drosophila histamine-gated Cl⁻ channel (HisCl1) expressed ectopically in the AIBs (cx15457) with 2 mM exogenous histamine (His) caused AIB hyperpolarization and locomotory phenotypes, but not paralysis. In contrast, increasing the histamine concentration to 10 mM caused paralysis that persisted for up to 24 hrs in the presence of histamine. Similarly, in the present Example, 2 mM histamine did not cause paralysis in wild type animals or in transgenic animals expressing HisCl1 in the AIBs (cx15457) on NGM plates (FIG. 8B). However, 2 mM histamine caused significance paralysis under the modified hypotonic assay conditions used in the present Example or when the histamine concentration was raised to 10 mM on NGM plates (FIGS. 8B-8C). Since the ablation of the AIBs does not cause paralysis, these results indicate that the partial inhibition of AIB signaling by partial hyperpolarization or the activation of Gαo signaling causes an imbalance in the locomotory circuit that results in a state of decision-making “confusion,” an inability to execute and sustain unidirectional movement and, ultimately, in cessation of locomotion (paralysis). Without wishing to be bound by theory, it is believed that any ligand that selectively unbalances AIB signaling can yield a similar locomotory phenotype and its target is a site for anthelmintic development.

Discussion

The monoamines 5-HT, DA, and TA each dramatically inhibit locomotion in C. elegans when applied exogenously at concentrations high enough to overcome the permeability barrier of the nematode cuticle, ultimately resulting in paralysis. In addition, monoamine-dependent locomotory paralysis is also observed in many parasitic nematodes, including Ascaris suum and Heterodera glycines. Using the C. elegans model, the receptors involved in monoamine-dependent locomotory inhibition have been identified and localized. The key receptors involved in 5-HT, DA, and TA inhibition each function at a different level in the locomotory circuit. For example, 5-HT-dependent paralysis in C. elegans involves the expression of the Gαₒ-coupled 5-HT₁-like receptor SER-4, and the 5-HT gated Cl⁻ channel MOD-1, in a limited number of interneurons, including the two AIBs. 5-HT₁-like agonists are seen to have anti-nematodal activity in vivo. The results of the present Example indicate that partial inhibition of the AIBs by activation of an endogenously expressed Gαₒ-coupled 5-HT₁-like receptor or 5-HT-gated Cl⁻ channel, or a heterologously expressed histamine-gated Cl⁻ channel, interferes with AIB signaling, causes “locomotory confusion” and ultimately paralysis. Animals with ablated AIBs are still motile and move efficiently, although their rates of spontaneous reversal are dramatically altered, indicating either that this partial inhibition differentially affects AIBs signaling to cause locomotory paralysis, or that the ablated animals have compensated for the loss of the AIBs.

This Example shows the utility of “chimeric” C. elegans, created by the heterologous, ectopic expression of key drug targets from parasitic nematodes, as a platform for agonist identification and anthelmintic compound screening. Although this Example is focused on inhibitory monoamine GPCRs and monoamine-gated ion channels, it can be expanded to any signaling molecules for which the appropriate mutant backgrounds can be prepared. Specific promoters are available for C. elegans muscles and most neurons; alternatively, specific promoters to other neurons can be generated using a Cre-Lox approach. This screening system has the ability to combine the individual pharmacologies of the receptors from different parasitic nematodes with the environment and accessory proteins necessary for functional expression. This becomes especially important because nematode-specific cell lines are not available, and the expression of nematode receptors in mammalian cells is quite variable and can require a host of additional modifications, including temperature shock to achieve expression.

Not only do transgenic C. elegans provide a promiscuous expression platform for distantly-related receptors, but these ectopically-expressed receptors are functional and maintain their ligand-receptor specificity, as highlighted above where only the transgenic animals expressing the human receptor were paralyzed by 8-OH-DPAT. The identification of DOP-3 as a secondary target in PAPP-dependent paralysis also validates the usefulness of transgenic C. elegans as a platform for drug target identification and anthelmintic compound screening. Although the current Example uses transgenic animals expressing the desired receptor as an extra-chromosomal array, stable lines can be readily constructed if required.

This screening platform also includes the nematode cuticle, a potential barrier to the entry of any anthelmintic, as well as a wide array of ABC transporters involved in drug efflux and resistance. The cuticle is made up of six layers, the epicuticle, external cortical, internal cortical, medial, fiber, and basal, as well as a carbohydrate-rich surface coat external to the epicuticle. Without wishing to be bound by theory, it is believed that the lipid-rich epicuticle layer is the key barrier to externally-applied drugs, especially water-soluble molecules (5-HT, TA, 80H-DPAT, etc.), and the reason for the high concentration required to cause paralysis under isotonic environment, i.e., on NGM agar plates. As mentioned above, although C. elegans cuticle is more impermeable than those of some parasitic nematodes, the permeability of the C. elegans cuticle can be manipulated by modifying incubation conditions and the availability of various mutant backgrounds. By incubating the animals in a salt-free, hypotonic environment, 5-HT paralyzes wild-type animals with an EC₅₀ of about 0.5 mM, in contrast with an EC₅₀ of about 12 mM on isotonic NGM agar plates. In addition, a number of C. elegans mutations that have increased cuticular permeability are also useful for enhancing small molecule screening against an array of medically-important targets, including those involved in locomotory paralysis. For example, many of the bus (bacterially swollen) mutations alter the cuticle and increase permeability. Indeed, as shown in FIGS. 3E-3F, it is possible to select specific cuticle mutants with permeabilities that mimic those of individual parasitic nematodes, providing a means to bypass the complicated and expensive process of culturing live parasites, at least during preliminary stages of agonist screening. This ability to alter cuticular permeability is certainly useful for agonist and anthelmintic compound identification, but in the case of the monoamines examined, relatively high concentrations of ligand are still required.

In summary, this Example identifies two key AIB interneurons that play a role in 5-HT-dependent paralysis, and indicates that partial inhibition of signaling from the neurons can cause “locomotory confusion,” and paralysis. In addition, this Example demonstrates and validates the utility of these “chimeric” C. elegans as a platform for agonist identification and anthelmintic compound screening.

### Example II—Anthelmintic Compounds

A library of compounds was screened for anthelmintic activity using the platform described above in Example I. Some of the results from this screening are shown in FIGS. 9A-9B. As seen from FIGS. 9A-9B, compound CD3-718 exhibited remarkable selectivity for C. elegans 5-HT₁ versus human 5-HT₁, causing paralysis in C. elegans animals expressing the nematode 5-HT₁ receptor, as well as wild type C. elegans, but not in animals expressing the human orthologue of the 5-HT₁ receptor, and not in animals that do not express any previously identified 5-HT receptors and do not respond to exogenous 5-HT (i.e., the 5-HT quint animals). Compound CD3-718 is therefore a highly selective anthelmintic compound.

### Example III—Flea, Heartworm, and Gastrointestinal Nematode Assays

Activity of the compounds CD3-718 and CD3-664, as well as a compound referred to as CD3-276, against flea (Ctenocephalides felis), heartworm (Dirofilaria immitis), and gastrointestinal nematode (Haemonchus contortus) was examined. The compound CD3-276 has the following structure:

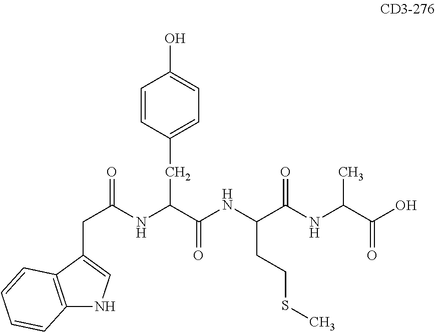 [SMILES: CSCCC(NC(=O)C(Cc1ccc(O)cc1)NC(=O)Cc1c[nH]c2ccccc12)C(=O)NC(C)C(=O)O]

Compound CD3-276 is also known as N-[(Indol-3)acetyl]-D-Tyrosyl-Methionyl-D-alanine, where the “D” specifies the unnatural (opposite enantiomer) amino acid.

For the flea (FF) membrane feed assay (adult), compounds were dissolved in DMSO and aliquots were added to citrated bovine blood in membrane covered wells warmed to 37° C. Adult fleas were newly emerged (3-7 days) and unfed. Feeding wells containing approximately 10 adult fleas were placed onto the treated blood wells, and the fleas were allowed to feed on the treated blood for 24 hours. Fleas were observed for knockdown and/or death at 24 hours. Each compound was tested at half-log intervals, and endpoint data was recorded as Minimum Effective Concentration (MEC) in μM. MEC is a subjective visual assessment of organism viability, and is the lowest dose to cause mortality ≧50%.

For the heartworm (HW) motility assay (microfilaria), compounds were dissolved in DMSO and half log dilutions were placed into individual wells. To each well was added approximately 200 Dirofilaria immitis microfilaria in a buffered media containing fetal bovine serum, Penicillin, and Streptomycin, resulting in a final concentration curve ranging from 100 μM to 1 nM. The microfilaria were observed with a light microscope for motility after a 72 hour incubation in a 5% CO₂, 37° C. incubator. The data reported is minimum efficacious dose (MED), which is the lowest concentration where there was a >50% decrease in motility compared to the DMSO controls.

For the gastrointestinal nematode (HC) motility assay (exsheathed L3), compounds were dissolved in DMSO and half log dilutions were placed into individual wells. To each well was added approximately 100 Haemonchus contortus exsheathed L3 larvae in a buffered nutrient media containing glucose and antibiotic panel resulting in a final concentration curve ranging from 100 μM to 1 nM. The larvae were observed with a light microscope for motility after 96 hours in a 37° C. incubator. The data reported is minimum efficacious dose (MED), which is the lowest concentration where there was a >50% decrease in motility compared to the DMSO controls.

The results of these assays are shown in Table 1.

TABLE 1
Results of FF, HW, and HC Assays

| Compound ID | FF (n = 2 mean) | HW (n = 2 mean) | HC (n = 2 mean) |
|---|---|---|---|
| CD3-718 | >100 μg/ml | >100 μg/ml | >100 μg/ml |
| CD3-664 | >100 μg/ml | 100 μg/ml | >100 μg/ml |
| CD3-276 | >100 μg/ml | >100 μg/ml | >100 μg/ml |

As seen from Table 1, CD3-664 was active at 100 μg/ml against heartworm. However, the compounds were inactive at the dose of 100 μg/ml against both fleas and H. contortus. CD3-718 and CD3-276 were inactive at the dose of 100 μg/ml against heartworm. These results indicate that CD3-664 is useful against heartworm.

Certain embodiments of the compounds, compositions, methods, and transgenic animals disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method of causing locomotory paralysis in a nematode leading to dysfunctional behavior or death, the method comprising:
exposing a nematode to an amount of a compound of Formula I effective to cause locomotory paralysis in the nematode leading to dysfunctional behavior or death: 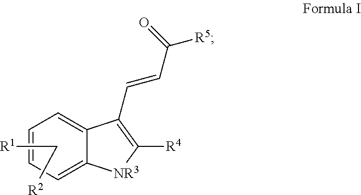
wherein:
R¹ and R² are either together or independently H, OH, or O-alkyl,
R³ and R⁴ are either together or independently H, alkyl, or aralkyl, and
R⁵ is alkyl, aryl, or aralkyl, wherein aryl includes heteroaryl and can be further substituted with one or more identical or different alkyl, O-alkyl, or OH groups;
or salts, stereoisomers, hydrates, solvates, racemates, polymorphs, or prodrugs thereof.

2. The method of claim 1, wherein:
R⁵ is either phenyl or pyridinyl; and
one of R¹ and R² is H, and the other of R¹ and R² is H, OH, or O-alkyl.

3. The method of claim 2, wherein:
R³ is H; and
R⁴ is either H or alkyl.

4. The method of claim 1, wherein the compound is compound CD3-718:

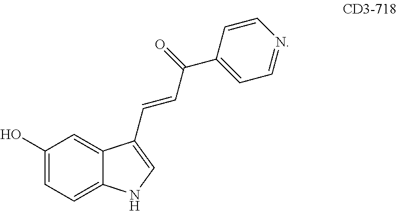 [SMILES: O=C(/C=C/c1c[nH]c2ccc(O)cc12)c1ccncc1]

5. The method of claim 1, wherein the compound comprises Formula II: 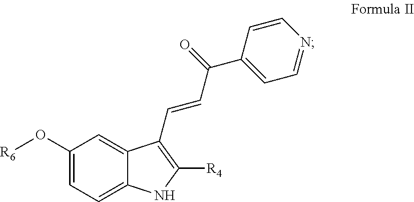
wherein R₄ and R₆ are each independently H or alkyl.

6. The method of claim 5, wherein the compound is compound CD3-719:

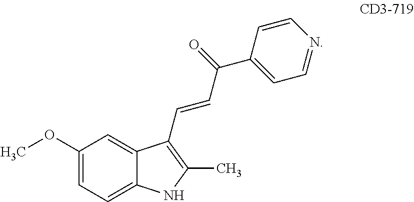 [SMILES: COc1ccc2[nH]c(C)c(/C=C/C(=O)c3ccncc3)c2c1]

7. The method of claim 5, wherein the compound is compound CD3-980:

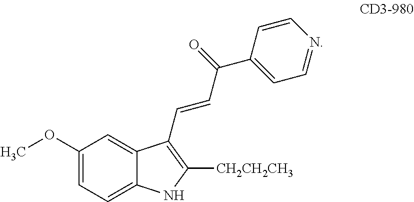

8. The method of claim 5, wherein the compound is CD3-984:

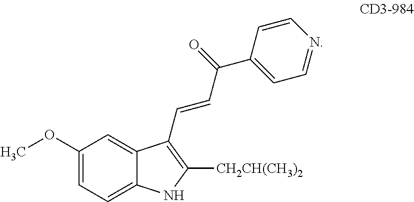

9. The method of claim 1, wherein the compound is CD4:

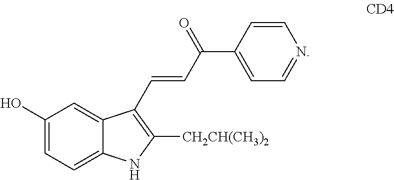

10. The method of claim 1, wherein the compound is CD3-664:

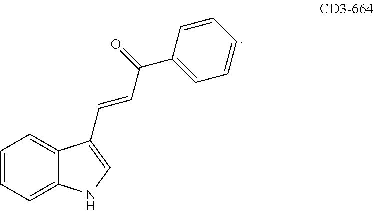 [SMILES: O=C(/C=C/c1c[nH]c2ccccc12)c1ccccc1]

11. The method of claim 1, wherein the nematode is present in soil or water, within a farm, within a drinking water supply, within a plant greenhouse, within an animal barn, within a human home, within a public building, within a manufacturing plant, within a restaurant, or within grocery store.

12. A method of treating a nematode infection in a living host, the method comprising:
administering to the host an effective amount of one or more compounds encompassed by Formula I to treat a nematode infection in the host: 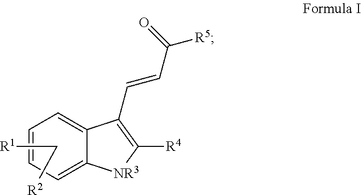
wherein:
R¹ and R² are either together or independently H, OH, or O-alkyl,
R³ and R⁴ are either together or independently H, alkyl, or aralkyl, and
R⁵ is alkyl, aryl, or aralkyl, wherein aryl includes heteroaryl and can be further substituted with one or more identical or different alkyl, O-alkyl, or OH groups;
or salts, stereoisomers, hydrates, solvates, racemates, polymorphs, or prodrugs thereof.

13. The method of claim 12, wherein:
R⁵ is either phenyl or pyridinyl; and
one of R¹ and R² is H, and the other of R¹ and R² is H, OH, or O-alkyl.

14. The method of claim 13, wherein:
R³ is H; and
R⁴ is either H or alkyl.

15. The method of claim 12, wherein the compound is compound CD3-718:

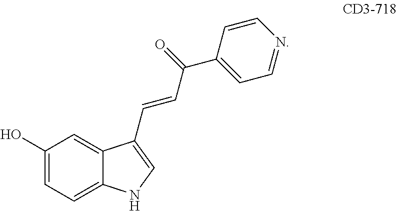 [SMILES: O=C(/C=C/c1c[nH]c2ccc(O)cc12)c1ccncc1]

16. The method of claim 12, wherein the compound is compound CD3-719:

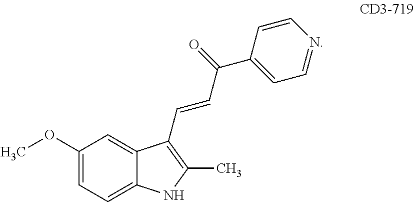 [SMILES: COc1ccc2[nH]c(C)c(/C=C/C(=O)c3ccncc3)c2c1]

17. The method of claim 12, wherein the compound is compound CD3-980:

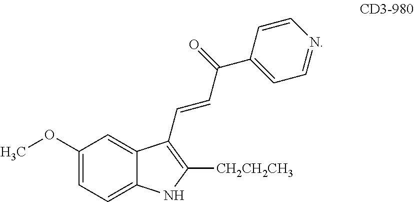

18. The method of claim 12, wherein the compound is CD3-984:

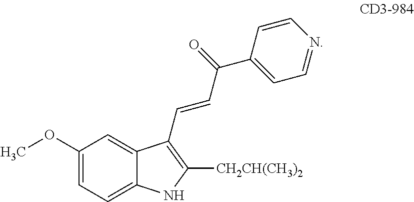

19. The method of claim 12, wherein the compound is CD4:

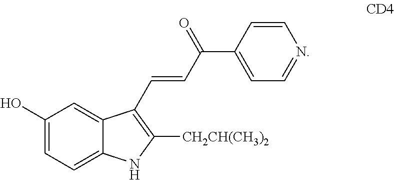

20. The method of claim 12, wherein the compound is CD3-664:

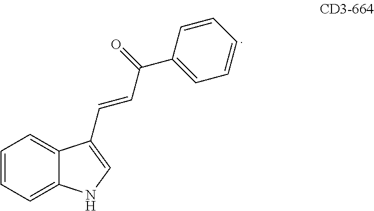 [SMILES: O=C(/C=C/c1c[nH]c2ccccc12)c1ccccc1]

21. A transgenic Caenorhabditis elegans comprising a 5-HT receptor null animal expressing a nematode, insect, animal, or human orthologue of a Gαₒ-coupled 5-HT₁-like receptor in cholinergic motor neurons.

22. The transgenic Caenorhabditis elegans of claim 21, wherein the Gαₒ-coupled 5-HT₁-like receptor comprises either a human 5-HT_{1A} receptor or nematode SER-4.

23. A method of assaying for antihelmintic selectivity, the method comprising:
administering a compound to a transgenic Caenorhabditis elegans of claim 21 that expresses a human orthologue of the Gαₒ-coupled 5-HT₁-like receptor, and observing whether the transgenic Caenorhabditis elegans exhibits locomotory paralysis; and
administering the compound to a wild type Caenorhabditis elegans or to a Caenorhabditis elegans expressing a nematode orthologue of the Gαₒ-coupled 5-HT₁-like receptor, and observing whether the wild type Caenorhabditis elegans or the Caenorhabditis elegans expressing a nematode orthologue of the Gαₒ-coupled 5-HT₁-like receptor exhibits locomotory paralysis to determine if the compound has selective antihelmintic activity;
wherein locomotory paralysis in the wild type Caenorhabditis elegans or the Caenorhabditis elegans expressing the nematode orthologue of the Gαₒ-coupled 5-HT₁-like receptor, and a lack or decreased amount of locomotory paralysis in the transgenic Caenorhabditis elegans, is indicative of selective antihelmintic activity.
